SEQUENCE LISTING (24 sequences)

SEQ ID NO: 1 - DNA, 987 bp, Zea mays
atgaagcggaagacgcggaacaagatcatcgtatggacgctggccctggctgcagtggcgattctggtgggcggcacgattgcgctggtgctcacggcggggacgtggaaggccaagataaagaagtcgcaggagaagatctgtaacaaggggtgggagtgctcggggagcaagtactgctgcaacgacaccatcaccgacttcttcaaggtgtaccagttcgagaacctcttcgccaagcgcaacacccccgtcgcgcacgccgtcgggttctgggactaccaggccttcatcaccgccgcggccctcttcgagccccaggggttctgcaccaccggcggcaagcagatgcagatgatggagctctgcgccttcctcgggcacgtcggcgccaagacttcatgtgggtacggcgtggcgaccggcgggccgacggcgtgggggctgtgctacaaccacgagatgagccccgaccagacctactgcgacaagacctacacccagtacccctgcgtcgagggcgccgagtactacggccgaggcgcgattcctgtctactggaactacaactacggcgctgccggtgacgggatcaaggcggatctgctccaccacccagagtacctggagcagaacgcgacgctggcattcatggcggcgatgtggcggtggatgacgccgatcaagaagagccagccgtcggcgcacgacgccttcgtgggcaactggaagcccaccaagaacgacacgctcagcaaacgcctgcctgggttcggcgccaccatgaacatactctacggcgagtcgatctgcggcaagggatacgtcgacgccatgaacgttataatctcgcactaccagtattaccttgatctcatgggcgtcggccgtgagcactctggcgacaaccgtgattgcgccgagcaggcaccgttcaacccctccagcccgacggatgaccagaagcagcagcaatcaggaagctaa

SEQ ID NO: 2 - protein, 328 aa, Zea mays
MKRKTRNKIIVWTLALAAVAILVGGTIALVLTAGTWKAKIKKSQEKICNKGWECSGSKYCCNDTITDFFKVYKFENLFAKRNTPVAHAVGFWDYQAFITAAALFEPQGFCTTGGKQMQMMELCAFLGHVGAKTSCGYGVATGGPTAWGLCYNHEMSPDQTYCDKTYTQYPCVEGAEYYGRGAIPVYWNYNYGAAGDGIKADLLHHPEYLEQNATLAFMAAMWRWMTPIKKSQPSAHDAFVGNWKPTKNDTLSKRLPGFGATMNILYGESICGKGYVDAMNVIISHYQYYLDLMGVGREHSGDNRDCAEQAPFNPSSPTDDQKQQQSGS

SEQ ID NO: 3 - protein, 328 aa, Zea mays
MKRKTRNKIIVWTLALAAVAILVGGTIALVLTAGTWKAKIKKSQEKICNKGWECSGSKYCCNDTITDFFKVYQFENLFAKRNTPVAHAVGFWDYQAFITAAALFEPQGFCTTGGKQMQMMELCAFLGHVGAKTSCGYGVATGGPTAWGLCYNHEMSPDQTYCDKTYTQYPCVEGAEYYGRGAIPVYWNYNYGAAGDGIKADLLHHPEYLEQNATLAFMAAMWRWMTPIKKSQPSAHDAFVGNWKPTKNDTLSKRLPGFGATMNILYGESICGKGYVDAMNVIISHYQYYLDLMGVGREHSGDNRDCAEQAPFNPSSPTDDQKQQQSGS

SEQ ID NO: 4 - protein, 328 aa, Sorghum bicolor
MKRKTRNKVIVWTLALAAAAILVGGTIALVLTAGTWKAQIKKSQEKICNKGWECSGSKYCCNDTITDFFKVYQFENLFAKRNTPVAHAVGFWDYQAFITAAALFEPQGFCTTGGKQMQMMELCAFLGHVGAKTSCGYGVATGGPTAWGLCYNHEMSPDQTYCDKTYTQWPCVEGAEYYGRGAIPVYWNYNYGAAGDGIKVDLLHHPEYLEQNATLAFMAAMWRWMTPIKKSQPSAHEAFVGTWKPTKNDTLSKRLPGFGATMNILYGESICGKGFVDSMNVIISHYQYYLDLMGVGREHSGDNRDCAEQLPFNPSSPTDDQKQQQSGS

SEQ ID NO: 5 - protein, 326 aa, Oryza sativa
MKRKTRNKIILTTLLVSAAAILIGGTVALILTAGTWKVKMKESREKICDKGWECSGSKYCCNDTITDFFKVYQFENLFSKRNSPVAHAVGFWDYQSFITAAALFEPLGFCTTGGKQMQMMELCAFLGHVGSKTSCGFGVATGGPTAWGLCYNHEMSPKEDYCDKTNLQYPCVEGAEYYGRGAIPVFWNYNYGAAGDGIHEDLLHHPEYLEQNATMAFMAAMWRWMTPMKKKQPSAHDVFVGNWKPTKNDTLAKRLPGFGATMNVLYGDQICGKGYIDDMNVIISHYQYYLDLMGVGREHSGDNRDCAEQAAFNPSYKKPDDQQQQS

SEQ ID NO: 6 - protein, 326 aa, Brachypodium distachyon
MKRKTRNKIVVTVLLVGAAAVLVGGTLALILTAGTWKMKVKEAHEKVCSKGWECSNSKYCCNQTISDFFKVYQFENLFAKRNTPIAKAVGFWDYQSFITASIPFQPQGFCTTGGKDMQMMELCAFLGHVGAKTSCGFGVATGGPTAWGLCYNREMSPEGIYCDDTYTQYPCVKGAEYYGRGAITVYWNYNYGAAGDGIKEDLLHHPEYLEQNATLAFQAAMWRWMTPIKKNQPSAHDAFVGNWKPTKNDTLSKRLPGFGATMNLLYGESICGRGFIDEMNVIISHYQYYLDLMGFGRERSGLNLDCAEQAPFNPTTKKADDQQPSG

SEQ ID NO: 7 - protein, 305 aa, Epipremnum aureum
MMCGRWAVAAAVLALMLAAGPAAAKKEKEKLCDKGWECKGPSKYCCNDTITDFFQVYQFENLFSKRNAPVAHAVGFWDYQAFITAAAVYQPLGFGTTGGKKMQMKEVAAFLGHVGSKTSCGYGVATGGPLAWGLCYNHEMSPSQDYCADNYQYPCTPGAQYHGRGALPVYWNYNYGVIGEGLNIDLLSHPEFLEQNATLAFQAAMWRWMNPMKKKQPSAHDVFVGNWKPTKNDTLSKRLPGFGATMNVLYGDLVCGQGYIDSMNNIISHYQYYLDLMGVGREFSGDNLDCAEQVAFNPSGKSATS

SEQ ID NO: 8 - protein, 280 aa, Elaeis guineensis
TREKEKVCNKGWECSGSIYCCNETITDYFQVYQFENLFSKRNAPVAHAVGFWDYQSFITAAAVYEPLGFGTTGGKQMGMKEVAAFLGHVGSKTSCGYGVATGGPLAWGLCYNHEMSPSQSYCADDYKYPCTPGAEYYGRGALPVYWNYNYGLVGEALKINLLNHPEYLEQNATMAFQAAMWRWMTPMKKKQPSAHDVFVGNWKPSKNDTLEKRLPGFGMTMNILYSDLVCGQGYIDSMNNIISHYQYYLDLMGIGREFSGDNVDCAEQKALNPSGKSASS

SEQ ID NO: 9 - protein, 309 aa, Elaeis guineensis
MAAKKVSSAIPMAISMLVFVLGVGVVAAKSKKEKLCTKGWECKASVYCCNQTISDLFQVYQFENLFSKRNTPVAHAVGFWDYQSFITASTAYQPQGFGTTGGKQMQMKEVAAFLGHVGSKTSCGYGVATGGPLAWGLCYNRELSPSKSYCEDSYVYPCTPGVEYYGRGALPVYWNYNYGTVGKELKVDLLSHPEYLEQNATLAFQAAMWRWMTPIKKKQPSAHDVFVGNWKPTKNDTLAKRLPGFGTTMNVLYGDLVCGQGSADSMNNIISHYQYYLDLMGVGRELSGDNLDCAEQQAFNPSAEKSSSS

SEQ ID NO: 10 - protein, 302 aa, Sorghum bicolor
MSGTRGAALLLLLLATLAAAVMAGGEKVCDKGWECSGSRFCCNETIGDFFKAYQFEELFPKRNSDLAHAAGFWDYKAFITAAALFEPRGFGTTGGKEMGMMEVAAFLGHVGAKTSCGYNEAPDGETAWGLCYNHELSPSQSYCDDSNELYPCVEGVEYYGRGALPVYWNYNYGIVGKGIKQDLLNHPELLEQNATLAFEAAIWRWMTPMKRKQPSAHDVFVGNWKPTKKDTLSKRYPGFGATMNILYGDAICGKGSTDKMNFIISHYQHYLGLMGVGRERSEDNLDCSDQVAFNPSSESSDF

SEQ ID NO: 11 - protein, 304 aa, Hordeum vulgare
MGMVRAASFLLLVALLAAADARRQKGGETACDKGWECSGSRFCCNETITDYFKAYQFEELFAQRNNSLAHAAGFWNYQAFITAASLFEPRGFGTTGGREMSMKEVAAFLGHVGAKTSCGYSLATGGSLAWGLCYNHEMSPSQSYCDDSNELYRCAEGVGYYGRGALPVYWNYNYGIVGKGIKQDLLNHPELLEQNATLAFEAAIWRWMTPMKRKQPSAHDAFVGNWKPTKKDTLSKRYPGFGVTMNILYGDAICGKGTTESMNAIISHYQHYLDLMGVGVQHSGDNLDCADQVPFNPSSKSPDS

SEQ ID NO: 12 - protein, 316 aa, Oryza sativa
MRTSRAAAAAASLPLLLLVALLVAAEGRRHKDGSGDEEAKACDKGWECSGSRFCCNDTITDYFKAYQFEELFAHRNDRSLAHAAGFWDYHAFITAAALFEPRGFGTTGGKEVGMKEVAAFLGHVGAKTSCGYSVATGGPLAWGLCYNHELSPSQSYCDNSNELYPCVEGVEYYGRGALPVYWNYNYGIIGQGIKQDLLNHPELLEQNATLAFEAAIWRWMTPMKRKQPSAHDVFVGNWKPTKNDTLSKRYPGFGATMNILYGDLICGQGSIDKMNVIVSHYQHYLDLMGVGSDKAGDNLDCADQVAFNPSSKNLDS

SEQ ID NO: 13 - protein, 316 aa, Oryza sativa
MRTSRAAAAAASLPLLLLVALLVAAEGRRHKDGGGDEEAKACDKGWECSGSRFCCNDTITDYFKAYQFEELFAHRNDRSLAHAAGFWDYHAFITAAALFEPRGFGTTGGKEVGMKEVAAFLGHVGAKTSCGYSVATGGPLAWGLCYNHELSPSQSYCDNSNELYPCVEGVEYYGRGALPVYWNYNYGIIGQGIKQDLLNHPELLEQNATLAFEAAIWRWMTPMKRKQPSAHDVFVGNWKPTKNDTLSKRYPGFGATMNILYGDLICGQGSIDKMNVIVSHYQHYLDLMGVGSDKAGDNLDCADQVAFNPSSKNLDS

SEQ ID NO: 14 - protein, 319 aa, Medicago truncatula
MAKLNAVVLLFAFFILLTTTVNGDESSNTKVQVKYKHGKKYCDKGWECKGWSIYCCNLTITDYFQTYQFENLFSKRNTPIAHAVGFWDYHSFINAASLFEPLGFGTTGNKTTQMMEIAAFLGHVGSKTSCGYGVATGGPLAWGLCYNHEMSPAQTYCDDYYKLTYPCTPGAEYYGRGAIPIYWNYNYGAAGEALKVNLLDHPEYIEQNATLAFQAAIWKWMTPIKKSQPSAHDAFVGNWKPTKNDTMENRVPGFGATMNILYGEGVCGQGDVDSMNNIVSHYLYYLDLLGVGRERAGTHDVLTCAEQRPFNPNTKLAAS

SEQ ID NO: 15 - protein, 321 aa, Vitis vinifera
MEAERSFLLIMAVAAMVVTVGGQGSGTKPLVKVLKGGLKVCDKGWECKGWSKYCCNQTVSDFFQTYQFENLFAKRNTPVAHAVGFWDYRSFITAAAVYQPHGFGTAGGKLMQMKEVAAFLGHVGSKTTCGYGVATGGPLAWGLCYNKEMSPSKSYCDDDYKYTYPCTPGVEYFGRGALPIYWNYNYGEAGEALKVDLLNHPEYIEQNATLAFQAAIWRWMTPVKKQQPSAHDVFVGTWKPTKNDTLAKRIPGFGATMNVLYGDSVCGQGDVDSMNNIVSHYQYYLDLMGVGREEAGPHEVLTCAEQEAFNPSSSSSSSSSS

SEQ ID NO: 16 - protein, 296 aa, Pisum sativum
GDDRNPKLQVKYKRGKKYCTQGWECNNWSIYCCNLTISDYFQTYHFENLFSKRNTPVAHAVGFWDYHSFINAAALFEPQGFGTTGNKTMQMMEIAAFLGHVGSKTSCGYGVATGGPTAWGLCYNHEMSPSQTYCDDYYKLTYPCTPGAEYYGRGAIPIYWNYNYGAAGEALKVNLLDHPEYIEQNATLAFQAAIWKWMTPVKKAQPSAHDAFVGNWKPTKNDTMGNRVPGFGATMNILYGEGVCGQGDVDSMNNIASHFLFYLDLLGVGRDKGGTHDVLTCAEQRPFNPNTKTASS

SEQ ID NO: 17 - protein, 321 aa, Lotus japonicus
MTSAKRTAVVFLTTLVIFLAVSSTVEGDDNTKTLVKYKHGEKYCDKGWECKGWSIYCCNLTISDYFQVYQFENLFSKRNTPVANAVGFWDYHSFITAAALFEPQGFGTTGNKTLQMMEIAAFLGHVGAKTSCGYGVATGGPLAWGLCYNHEMSPSQKYCDEYYKLTYPCTPGADYYGRGAIPIYWNYNYGAVGEALKVNLLDHPEYIEQNATLAFLAAIWRWMTPNKKYQPSAHDIFVGKWKPSKNDTMEKRIPGFGSTMNVLYGESVCGQGDIDPMNVIISHYLYYLDLLGVGRETAGPHEFLSCAEQVAFNPSTKAASS

SEQ ID NO: 18 - protein, 329 aa, Glycine max
MMETTAKRNAVVAPIAALLLVVLLAASVVVEAQENSDVKTLVKHMHRKKYCDKGWECKGWSIYCCNLTITDYFQTYQFENLFSKRNSPVAHAVGFWGYHSFIAAAALFEPQGFGTTGNKTMQMMEIAAFLGHVGSKTSCGYGVATGGPLAWGLCYNHEMSPMQSYCDDYFKLTYPCTPGAEYYGRGAIPIFWNYNYGAVGEALKIDLLSHPEYIEQNATLAFQAAIWRWMTPMKKSQPSAHDAFVGNWKPSKDDTMENRLPGFGTTMNILYGDGVCGQGDVDSMNNIVSHYQYYLDLLGVGREQAGPHEILTCAEQVPFNPSSKPASSS

SEQ ID NO: 19 - protein, 326 aa, Glycine max
MMMEMAKRNAVVAALAALVLLAASVAVDAQENSDVKTLVKYKHGKKYCDKGWECKGWSIYCCNLTITDYFQTYQFENLFSKRNSPVAHAVGFWDYHSFIAAAALFEPEGFGTTGNKTMQMMEIAAFFGHVGSKTSCGYGVATGGPLAWGLCYNHEMSPMQSYCDDYFKLTYPCTPGADYYGRGAIPIFWNYNYGAVGEALKIDLLSHPEYIEQNATLAFQAAIWRWMTPMKKSQPSAHDAFVGSWKPTKNDTMENRVPGFGTTMNILYGDGVCGQGDVDSMNNIVSHYLYYLDLLGVGREQAGPHDILTCAEQVPFNPSSKPASSS

SEQ ID NO: 20 - protein, 321 aa, Arabidopsis thaliana
MVTIRSGSIVILVLLAVSFLALVANGEDKTIKVKKVRGNKVCTQGWECSWWSKYCCNQTISDYFQVYQFEQLFSKRNTPIAHAVGFWDYQSFITAAALFEPLGFGTTGGKLMGQKEMAAFLGHVASKTSCGYGVATGGPLAWGLCYNREMSPMQSYCDESWKFKYPCSPGAEYYGRGALPIYWNFNYGAAGEALKADLLNHPEYIEQNATLAFQAAIWRWMTPIKRAQPSAHDIFVGNWKPTKNDTLSKRGPTFGSTMNVLYGEYTCGQGSIDPMNNIISHYLYFLDLMGIGREDAGPNDELSCAEQKPFNPSTVPSSSSS

SEQ ID NO: 21 - protein, 321 aa, Ricinus communis
MEKWIRCFMIIAALLVANWGLLTNGDDSEKTVVKMVRGKKVCTRGWECASWSEYCCNQTISDFFQTYQFENLFSKRNAPVAHAIGFWDYQSFITASALFQPLGFCTTGGKLMQMKELAAFFSHVGSKTSCGYGVATGGPLAYGLCYNREMSPSQSYCDDFYKFTYPCTPGAEYYGRGALPIYWNYNYGATGEALKVNLLDHPEYIEQNATLAFQAAIWRWMTPMKKSQPSAHDVFVGNWKPTKNDTLSKRIPGFGTTMNILYGDLVCGQGDIDGMNNIVSHYLYYLDLMGVGREEAGPHEYLTCAEQKAFNPSFLDPASSS

SEQ ID NO: 22 - protein, 321 aa, Arabidopsis thaliana
MVTIRSGSIVILVLLAVSFLALVANGEDKTIKVKKVRGNKVCTQGWECSWWSKYCCNQTISDYFQVYQFEQLFSKRNTPIAHAVGFWDYQSFITAAALFEPLGFGTTGGKLMGQKEMAAFLGHVASKTSCGYGVATGGPLAWGLCYNREMSPMQSYCDESWKFKYPCSPGAEYYGRGALPIYWNFNYGAAGEALKADLLNHPEYIEQKATLAFQAAIWRWMTPIKRAQPSAHDIFVGNWKPTKNDTLSKRGPTFGSTMNVLYGEYTCGQGSIDPMNNIISHYLYFLDLMGIGREDAGPNDELSCAEQKPFNPSTVPSSSSS

SEQ ID NO: 23 - protein, 321 aa, Arabidopsis lyrata
MVTIRSGSIVILVLLAVSFLALVANGEDKTIKVKKVRGERVCTQGWECSWWSKYCCNQTISDYFQVYQFEQLFSKRNTPIAHAVGFWDYQSFITAAALYEPLGFGTTGGKLQGQKEMAAFLGHVASKTSCGYGVATGGPLAWGLCYNREMSPMQSYCDESWKFKYPCSPGAEYYGRGALPIYWNFNYGAAGEALKADLLNHPEYIEQNATLAFQAAIWRWMTPIKKYQPSAHDIFVGNWKPTKNDTLSKRGPTFGSTMNVLYGEYTCGQGSIDPMNNIISHYLYFLDLLGIGREDAGPNEELSCAEQKPFNPSTVPSSSSS

SEQ ID NO: 24 - protein, 319 aa, Acacia koa
MKVPRSVIFLLLLALVLLVASADESNTKPLVKYHKGKKLCDKGWECKGWSIYCCNLTITDYFQPYQFENLFSKRNSPVAHAVGFWDYHSFITAAAVYEPLGFGTTGNKTMQMKEIAAFLAHVGSKTSCGYGVATGGPFAWGLCYSHEMSPSQSYCDDYFKYTYPCAPGADYYGRGALPIFWNYNYGAAGEALKVDLLSHPEYVEQNATLAFQAAIWRWMTPIKKKQPSAHDAFVGSWKPTKNDTIANRLPGFGTTMNILYGDGVCGQGDVDSMNNIVSHYLYYLDLLGVGREDAGPHELLTCAEQVPFNPSSSTATAAS

(19) United States
(12) Patent Application Publication
(10) Pub. No.: US 2016/0002658 A1
(43) Pub. Date: Jan. 7, 2016

(54) COMPOSITIONS AND METHODS TO ENHANCE MECHANICAL STALK STRENGTH IN PLANTS

(71) Applicant: PIONEER HI-BRED INTERNATIONAL, INC., Johnston, IA (US)

(72) Inventor: Dilbag S. Multani, Urbandale, IA (US)

(21) Appl. No.: 14/770,644

(22) PCT Filed: Mar. 10, 2014
(86) PCT No.: PCT/US14/22247

§ 371 Date: Aug. 26, 2015

Related U.S. Application Data
(60) Provisional application No. 61/775,801, filed on Mar. 11, 2013.

(51) Int. Cl. C12N 15/82; C12N 9/42

(52) U.S. Cl. CPC C12N 15/8262; C12N 9/2442; C12Y 302/01014

(57) ABSTRACT

Isolated polynucleotides and polypeptides and recombinant DNA constructs useful for enhancing mechanical stalk strength in plants, compositions (such as plants or seeds) comprising these recombinant DNA constructs, and methods utilizing these recombinant DNA constructs. The recombinant DNA construct comprises a polynucleotide operably linked to a promoter that is functional in a plant, wherein said polynucleotide encodes a CTL1 polypeptide.

### CROSS REFERENCE TO RELATED APPLICATIONS

This application claims the benefit of U.S. Provisional Application No. 61/775,801, filed Mar. 11, 2013, the entire contents of which are hereby incorporated by reference.

### FIELD OF THE DISCLOSURE

The field of disclosure relates to plant breeding and genetics and in particular, to recombinant DNA constructs useful in enhancing mechanical stalk strength in plants.

### BACKGROUND OF THE DISCLOSURE

In maize, stalk lodging, or stalk breakage, accounts for significant annual yield losses in the United States. During a maize plant's vegetative growth phase, rapid growth weakens cell walls, making stalk tissue brittle and increasing the propensity for stalks to snap when exposed to strong, sudden winds and/or other weather conditions. This type of stalk lodging, called green snap or brittle snap, typically occurs at the V5 to V8 stage, when the growing point of a maize plant is emerging from the soil line, or at the V12 to R1 stage, about two weeks prior to tasseling and until just after silking. Another type of stalk lodging, late season stalk lodging occurs near harvest when the stalk cannot support the weight of the ear. Factors that weaken the stalk during late season include insect attack, such as the European corn borer tunneling into stalk and ear shanks, and infection by pathogens such as Colletotrichum graminicola, the causative agent in Anthracnose stalk rot. Adverse fall weather conditions also contribute to late season stalk lodging.

The mechanical strength of the maize stalk plays a major role in a plant's resistance to all types of stalk lodging, and therefore, is of great value to the farmer. Enhancing overall mechanical stalk strength in maize will make stalks stronger during both vegetative development and late season, thereby reducing yield and grain quality losses. Moreover, maize plants with enhanced mechanical stalk strength can remain in the field for longer periods of time, allowing farmers to delay harvest, if necessary.

### SUMMARY OF THE DISCLOSURE

In one embodiment, a plant comprising in its genome a recombinant DNA construct comprising a polynucleotide operably linked to at least one regulatory element, wherein said polynucleotide encodes a polypeptide having an amino acid sequence of at least 50%, 60%, 70%, 80%, 85%, 90%, 95% or 100% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24, and wherein said plant exhibits enhanced mechanical stalk strength when compared to a control plant not comprising said recombinant DNA construct.

In another embodiment, the plants may be selected from the group consisting of: Arabidopsis, maize, soybean, sunflower, sorghum, canola, wheat, alfalfa, cotton, rice, barley, millet, sugar cane and switchgrass.

In another embodiment, the present disclosure includes seed of any of the plants of the present disclosure, wherein said seed comprises in its genome a recombinant DNA construct comprising a polynucleotide operably linked to at least one regulatory element, wherein said polynucleotide encodes a polypeptide having an amino acid sequence of at least 50%, 60%, 70%, 80%, 85%, 90%, 95% or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, or 24, and wherein a plant produced from said seed exhibits enhanced mechanical stalk strength when compared to a control plant not comprising said recombinant DNA construct.

In another embodiment, a method of enhancing mechanical stalk strength in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50%, 60%, 70%, 80%, 85%, 90%, 95% or 100% sequence identity, based on the Clustal V method of alignment, when compared to SEQ ID NO: SEQ ID NO:2, 3, 4, 5, 6, 7, 8, 9, 10, 11, 12, 13, 14, 15, 16, 17, 18, 19, 20, 21, 22, 23, or 24; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits enhanced mechanical stalk strength when compared to a control plant not comprising the recombinant DNA construct.

In another embodiment, a method of selecting for enhanced mechanical stalk strength in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50%, 60%, 70%, 80%, 85%, 90%, 95% or 100% sequence identity, based on the Clustal V method of alignment, when compared to SEQ ID NO: SEQ ID NO:2, 3, 4, 5, 6, 7, 8, 9, 10, 11, 12, 13, 14, 15, 16, 17, 18, 19, 20, 21, 22, 23, or 24; (b) growing the transgenic plant of part (a); and (c) selecting the transgenic plant of part (b) with enhanced mechanical stalk strength compared to a control plant not comprising the recombinant DNA construct.

In another embodiment, in any of the methods of the present disclosure, the plant may be selected from the group consisting of: Arabidopsis, maize, soybean, sunflower, sorghum, canola, wheat, alfalfa, cotton, rice, barley, millet, sugar cane and switchgrass.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows representative images of bk4 mutant plants (bk4-1 allele) alongside their WT (wild-type) sibs. A) Stalks B) Roots

FIG. 2 is a graph showing the average internode length and stalk diameter of bk4 mutants as compared to their het or wt sibs.

FIG. 3 is a graph showing the mechanical stalk strength of bk4 mutants as compared to their het or WT-sibs.

FIG. 4 shows a schematic representation of the maize Bk4 (also known as ZmCtl1) gene and the positions of the Mu insertions in the bk4-1, bk4-2, and bk4-3 mutant lines. Exons are represented by filled rectangles and introns are represented by lines.

FIG. 5 shows RT-PCR analysis using ten days old seedlings and primers specific to Zm-Ct/1. Results showed missing transcripts in the homozygous mutants when compared to their WT-sibs.

FIG. 6 is a graph showing the expression of maize CM gene in different tissues as compiled from an internal proprietary MPSS database.

FIG. 7 is a graph showing the sugar composition of stalks of bk4 mutant plants and their WT-sibs (from darkest to lightest is arabinose %, galactose %, glucose %, xylose %, and mannose %).

FIG. 8 is a graph showing differences in p-coumaric and ferulic acid levels in dried stalk tissue in Bk4 mutant and WT-sib maize plants.

FIG. 9 shows differences in lignin localization in maize stems between WT-sibs and bk4 mutants. There is a significant reduction in lignin staining in the rind collenchyma cells and bundle fibers throughout the stem of bk4 mutants as compared to their WT-sibs. Deformed bundles in the pith of bk4 mutant are common.

FIGS. 10A-10F present an alignment of the amino acid sequences of the polypeptides set forth in SEQ ID NOs:2-24.

FIGS. 11A and 11B present the percent sequence identities and divergence values for each sequence pair presented in FIGS. 10A-10F.

FIG. 12 shows that T1 plants that are overexpressing ZmCtl1 have increased maximum flexural load as compared to negative controls.

FIG. 13 shows that T1 plants that are overexpressing ZmCtl1 increase the average ferulic acid content as compared to negative controls.

FIG. 14 shows that T1 plants that are overexpressing ZmCtl1 are similar to negative controls with respect to p-coumaric acid levels.

FIG. 15 shows that T1 plants that are overexpressing ZmCtl1 are similar to negative controls with respect to glucose and xylose composition.

FIG. 16 shows that T1 plants that are overexpressing ZmCtl1 are similar to negative controls with respect to arabinose, galactose, and mannose compositions.

FIG. 17 shows that T1 plants that are overexpressing ZmCtl1 are similar to negative controls with respect to % xylose/% arabinose ratios.

SEQ ID NO:1 is the nucleotide sequence of the genomic wild-type Zea mays Ctl1.

SEQ ID NO:2 is the amino acid sequence of the wild-type Zea mays CTL1 (ZmCTL1) protein.

SEQ ID NO:3 is the amino acid sequence of an uncharacterized protein from Zea mays (NCBI GI No. 226500888).

SEQ ID NO:4 is the amino acid sequence of a hypothetical protein from Sorghum bicolor (NCBI GI No. 242045186).

SEQ ID NO:5 is the amino acid sequence of a hypothetical protein from Oryza sativa (NCBI GI No. 115479911).

SEQ ID NO:6 is the amino acid sequence of a chitinase-like protein 1-like from Brachypodium distachyon (NCBI GI No. 357159137).

SEQ ID NO:7 is the amino acid sequence of a putative chitinase from Epipremnum aureum (NCBI GI No. 283046278).

SEQ ID NO:8 is the amino acid sequence of a class I chitinase from Elaeis guineensis (NCBI GI No. 342151641).

SEQ ID NO:9 is the amino acid sequence of a chitinase-like protein from Elaeis guineensis (NCBI GI No. 409191689).

SEQ ID NO:10 is the amino acid sequence of a hypothetical protein from Sorghum bicolor (NCBI GI No. 242082217).

SEQ ID NO:11 is the amino acid sequence of a predicted protein from Hordeum vulgare (NCBI GI No. 326529205).

SEQ ID NO:12 is the amino acid sequence of a hypothetical protein from Oryza sativa (NCBI GI No. 115477370).

SEQ ID NO:13 is the amino acid sequence of a hypothetical protein from Oryza sativa (NCBI GI No. 125562231).

SEQ ID NO:14 is the amino acid sequence of an endochitinase from Medicago truncatula (NCBI GI No. 357502783).

SEQ ID NO:15 is the amino acid sequence of a chitinase-like protein 2 from Vitis vinifera (NCBI GI No. 225431904).

SEQ ID NO:16 is the amino acid sequence of a class1 chitinase from Pisum sativum (NCBI GI No. 37051096).

SEQ ID NO:17 is the amino acid sequence of an unknown protein from Lotus japonicas (NCBI GI No. 388492432).

SEQ ID NO:18 is the amino acid sequence of an uncharacterized protein from Glycine max (NCBI GI No. 363807428).

SEQ ID NO:19 is the amino acid sequence of a chitinase-like protein 1-like isoform 1 from Glycine max (NCBI GI No. 356526631).

SEQ ID NO:20 is the amino acid sequence of a chitinase-like protein 1 from Arabidopsis thaliana (NCBI GI No. 15221283).

SEQ ID NO:21 is the amino acid sequence of a putative chitinase from Ricinus communis (NCBI GI No. 255549220).

SEQ ID NO:22 is the amino acid sequence of a hypothetical protein from Arabidopsis thaliana (NCBI GI No. 225897882).

SEQ ID NO:23 is the amino acid sequence of a pom-pom1 protein from Arabidopsis lyrata (NCBI GI No. 297848858).

SEQ ID NO:24 is the amino acid sequence of a class Ib chitinase from Acacia koa (NCBI GI No. 425886500).

The sequence descriptions and Sequence Listing attached hereto comply with the rules governing nucleotide and/or amino acid sequence disclosures in patent applications as set forth in 37 C.F.R. §1.821 1.825.

The Sequence Listing contains the one letter code for nucleotide sequence characters and the three letter codes for amino acids as defined in conformity with the IUPAC IUBMB standards described in Nucleic Acids Res. 13:3021 3030 (1985) and in the Biochemical J. 219 (No. 2):345 373 (1984) which are herein incorporated by reference. The symbols and format used for nucleotide and amino acid sequence data comply with the rules set forth in 37 C.F.R. §1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

Plant chitinases are enzymes that presumably hydrolyze chitin, a biopolymer of GlcNAc in a β-1,4 linkage. Plant chitinases are grouped into sixe different classes based on sequence similarity, with the two most common classes being class I and class II. Class I chitinases possess a conserved N-terminal cysteine-rich lectin domain and are believed to be essential for normal plant growth and development.

“CTL1 polypeptide” is a member of the class I plant chitinases. The terms “BK4” and “CTL1” are used interchangeably herein.

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

The term “enhanced mechanical stalk strength” refers to an increase in the ability of a plant to resist breakage when a mechanical force is applied to the plant. In general, plants with “enhanced mechanical stalk strength” are resistant to stalk lodging and have mechanically stronger stalks. The term “enhanced” relates to the degree of physical strength and/or the degree of resistance to breakage.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Sequence alignments and percent identity calculations may be determined using a variety of comparison methods designed to detect homologous sequences including, but not limited to, the Megalign® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning now to the embodiments:

Embodiments include recombinant DNA constructs useful for conferring enhanced mechanical strength, compositions (such as plants or seeds) comprising these recombinant DNA constructs, and methods utilizing these recombinant DNA constructs.

Isolated Polynucleotides and Polypeptides:

The present invention includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, or 24, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs of the present invention. The polypeptide is preferably a CTL1 polypeptide. The CTL1 polypeptide preferably has chitinase I activity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, or 24, and combinations thereof. The polypeptide is preferably a CTL1 polypeptide. The CTL1 polypeptide preferably has chitinase I activity

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:1, and combinations thereof; or (ii) a full complement of the nucleic acid sequence of (i). Any of the foregoing isolated polynucleotides may be utilized in any recombinant DNA constructs of the present invention. The isolated polynucleotide preferably encodes a CTL1 polypeptide. The CTL1 polypeptide preferably has chitinase I activity.

An isolated polynucleotide comprising a nucleotide sequence, wherein the nucleotide sequence is hybridizable under stringent conditions with a DNA molecule comprising the full complement of SEQ ID NO:1. The isolated polynucleotide preferably encodes a CTL1 polypeptide. The CTL1 polypeptide preferably has chitinase I activity.

An isolated polynucleotide comprising a nucleotide sequence, wherein the nucleotide sequence is derived from SEQ ID NO:1 by alteration of one or more nucleotides by at least one method selected from the group consisting of: deletion, substitution, addition and insertion. The isolated polynucleotide preferably encodes a CTL1 polypeptide. The CTL1 polypeptide preferably has chitinase I activity.

An isolated polynucleotide comprising a nucleotide sequence, wherein the nucleotide sequence corresponds to an allele of SEQ ID NO:1.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current invention may also be a protein which comprises an amino acid sequence comprising deletion, substitution, insertion and/or addition of one or more amino acids in an amino acid sequence presented in SEQ ID NO: 3, 4, 5, 6, 7, 8, 9, 10, 11, 12, 13, 14, 15, 16, 17, 18, 19, 20, 21, 22, 23, or 24. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present invention may also be a protein which is encoded by a nucleic acid comprising a nucleotide sequence comprising deletion, substitution, insertion and/or addition of one or more nucleotides in the nucleotide sequence of SEQ ID NO:1. Nucleotide deletion, substitution, insertion and/or addition may be accomplished by site-directed mutagenesis or other techniques as mentioned above.

The protein of the present invention may also be a protein which is encoded by a nucleic acid comprising a nucleotide sequence hybridizable under stringent conditions with the complementary strand of the nucleotide sequence of SEQ ID NO:1.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs:

In one aspect, the present invention includes recombinant DNA constructs.

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 3, 4, 5, 6, 7, 8, 9, 10, 11, 12, 13, 14, 15, 16, 17, 18, 19, 20, 21, 22, 23, or 24, and combinations thereof; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:1, and combinations thereof; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide encodes a class I chitinase. The class I chitinase may be from Arabidopsis thaliana, Zea mays, Glycine max, Glycine tabacina, Glycine soja, Glycine tomentella, Oryza sativa, Brassica napus, Sorghum bicolor, Saccharum officinarum, Triticum aestivum, Brachypodium distachyon, Epipremnum aureum, Elaeis guineensis, Hordeum vulgare, Medicago truncatula, Vitis vinifera, Pisum sativum, Lotus japonicus, Ricinus communis, Arabidopsis lyrata, or Acacia koa.

It is understood, as those skilled in the art will appreciate, that the invention encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

Regulatory Sequences:

A recombinant DNA construct of the present invention may comprise at least one regulatory sequence.

A regulatory sequence may be a promoter.

A number of promoters can be used in recombinant DNA constructs of the present invention. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance mechanical stalk strength in plants. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the invention, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present invention which causes the desired temporal and spatial expression.

Promoters which are seed or embryo-specific and may be useful in the invention include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current invention include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and Ciml which is specific to the nucleus of developing maize kernels. Ciml transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present invention in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use in the current invention may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664),

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

Any plant can be selected for the identification of regulatory sequences and CTL1 genes to be used in recombinant DNA constructs and other compositions (e.g. transgenic plants, seeds and cells) and methods of the present invention. Examples of suitable plants for the isolation of genes and regulatory sequences and for compositions and methods of the present invention would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, a vine, watermelon, wheat, yams, and zucchini.

Compositions:

A composition of the present invention includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present invention is a plant comprising in its genome any of the recombinant DNA constructs of the present invention (such as any of the constructs discussed above). Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct. These seeds can be grown to produce plants that would exhibit enhanced mechanical stalk strength, or used in a breeding program to produce hybrid seed, which can be grown to produce plants that would exhibit enhanced mechanical stalk strength. The seeds may be maize seeds.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

The recombinant DNA construct may be stably integrated into the genome of the plant.

Particular embodiments include but are not limited to the following:

1. A plant (for example, a maize, rice or soybean, plant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24, and wherein said plant exhibits enhanced mechanical stalk strength when compared to a control plant not comprising said recombinant DNA construct.

2. A plant (for example, a maize, rice or soybean plant) comprising in its genome a recombinant DNA construct comprising a polynucleotide operably linked to at least one regulatory sequence, wherein said polynucleotide encodes a CTL1 polypeptide, and wherein said plant exhibits enhanced mechanical stalk strength when compared to a control plant not comprising said recombinant DNA construct.

3. A plant (for example, a maize, rice or soybean plant) comprising in its genome a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1; or (b) derived from SEQ ID NO:1 by alteration of one or more nucleotides by at least one method selected from the group consisting of: deletion, substitution, addition and insertion; and wherein said plant exhibits enhanced mechanical stalk strength, when compared to a control plant not comprising said recombinant DNA construct.

4. A plant (for example, a maize, rice or soybean plant) comprising in its genome a polynucleotide (optionally an endogenous polynucleotide) operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24, and wherein said plant exhibits enhanced mechanical stalk strength when compared to a control plant not comprising the recombinant regulatory element. The at least one heterologous regulatory element may comprise an enhancer sequence or a multimer of identical or different enhancer sequences. The at least one heterologous regulatory element may comprise one, two, three or four copies of the CaMV 35S enhancer.

5. Any progeny of the plants in the embodiments described herein, any seeds of the plants in the embodiments described herein, any seeds of progeny of the plants in embodiments described herein, and cells from any of the above plants in embodiments described herein and progeny thereof.

In any of the embodiments described herein, the CTL1 polypeptide may be from Arabidopsis thaliana, Zea mays, Glycine max, Glycine tabacina, Glycine soja, Glycine tornentella, Oryza sativa, Brassica napus, Sorghum bicolor, Saccharum officinarum, Triticum aestivum, Brachypodium distachyon, Epipremnum aureum, Elaeis guineensis, Hordeum vulgare, Medicago truncatula, Vitis vinifera, Pisum sativum, Lotus japonicus, Ricinus communis, Arabidopsis lyrata, or Acacia koa.

In any of the embodiments described herein, the recombinant DNA construct may comprise at least a promoter functional in a plant as a regulatory sequence.

One of ordinary skill in the art is familiar with protocols for evaluating mechanical stalk strength in plants. Some methods involve the measurement of stalk diameter or dry weight per plant, while others can utilize an Instron™ machine or other similar crushing device to assess the load needed to break a stalk. The three point bend test is often used in conjunction with an Instron™ machine or other similar crushing device, and mechanical stalk strength values obtained from the three-point bend test have shown to be highly correlated to lodging scores assigned based on field observations. Still another method can involve the use of a stalk-penetrating device.

In addition, any method that uses a device to accurately reproduce wind forces, in order to select plants with increased mechanical stalk strength in the field, can be utilized for the characterization of mechanical stalk strength in maize plants. A device and method used to screen for selected wind-resistance traits in maize, including stalk strength, are described in patent application US200710125155 (published Jun. 6, 2007). When this device and method are used, the unit of measure is the number or percentage of plants that have lodged, or broken, stalks (or, alternatively, the number or percentage of plants that do not lodge).

One of ordinary skill in the art would readily recognize a suitable control or reference plant to be utilized when assessing or measuring a phenotype (e.g. mechanical stalk strength) of a transgenic plant in any embodiment of the present invention in which a control plant is utilized (e.g., compositions or methods as described herein). For example, by way of non-limiting illustrations:

1. Progeny of a transformed plant which is hemizygous with respect to a recombinant DNA construct, such that the progeny are segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).

2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).

4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring a phenotype (e.g. mechanical stalk strength) of a transgenic plant would not include a plant that had been previously selected, via mutagenesis or transformation, for the desired phenotype.

Methods:

Methods include but are not limited to methods for enhancing mechanical stalk strength in a plant, methods for evaluating mechanical stalk strength in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane or sorghum. The seed may be a maize or soybean seed, for example, a maize hybrid seed or maize inbred seed.

Methods include but are not limited to the following:

A method for transforming a cell (or microorganism) comprising transforming a cell (or microorganism) with any of the isolated polynucleotides or recombinant DNA constructs of the present invention. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs of the present invention and regenerating a transgenic plant from the transformed plant cell. The invention is also directed to the transgenic plant produced by this method, and transgenic seed obtained from this transgenic plant. The transgenic plant obtained by this method may be used in other methods of the present invention.

A method for isolating a polypeptide of the invention from a cell or culture medium of the cell, wherein the cell comprises a recombinant DNA construct comprising a polynucleotide of the invention operably linked to at least one regulatory sequence, and wherein the transformed host cell is grown under conditions that are suitable for expression of the recombinant DNA construct.

A method of altering the level of expression of a polypeptide of the invention in a host cell comprising: (a) transforming a host cell with a recombinant DNA construct of the present invention; and (b) growing the transformed host cell under conditions that are suitable for expression of the recombinant DNA construct wherein expression of the recombinant DNA construct results in production of altered levels of the polypeptide of the invention in the transformed host cell.

A method of enhancing mechanical stalk strength in a plant, comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, or 24; and (b) regenerating a transgenic plant from the regenerable plant cell after step (a), wherein the transgenic plant comprises in its genome the recombinant DNA construct and exhibits enhanced mechanical stalk strength when compared to a control plant not comprising the recombinant DNA construct. The method may further comprise (c) obtaining a progeny plant derived from the transgenic plant, wherein said progeny plant comprises in its genome the recombinant DNA construct and exhibits enhanced mechanical stalk strength when compared to a control plant not comprising the recombinant DNA construct.

A method of enhancing mechanical stalk strength in a plant, the method comprising: (a) introducing into a regenerable plant cell a recombinant DNA construct comprising a polynucleotide operably linked to at least one regulatory element, wherein said polynucleotide comprises a nucleotide sequence, wherein the nucleotide sequence is: (a) hybridizable under stringent conditions with a DNA molecule comprising the full complement of SEQ ID NO:1; or (b) derived from SEQ ID NO:1 by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits enhanced mechanical stalk strength when compared to a control plant not comprising the recombinant DNA construct. The method may further comprise (c) obtaining a progeny plant derived from the transgenic plant, wherein said progeny plant comprises in its genome the recombinant DNA construct and exhibits enhanced mechanical stalk strength, when compared to a control plant not comprising the recombinant DNA construct.

A method of selecting for (or identifying) enhanced mechanical stalk strength in a plant, comprising (a) obtaining a transgenic plant, wherein the transgenic plant comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 3, 4, 5, 6, 7, 8, 9, 10, 11, 12, 13, 14, 15, 16, 17, 18, 19, 20, 21, 22, 23, or 24; (b) obtaining a progeny plant derived from said transgenic plant, wherein the progeny plant comprises in its genome the recombinant DNA construct; and (c) selecting (or identifying) the progeny plant with enhanced mechanical stalk strength compared to a control plant not comprising the recombinant DNA construct.

In another embodiment, a method of selecting for (or identifying) enhanced mechanical stalk strength in a plant, comprising: (a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24; (b) growing the transgenic plant of part (a); and (c) selecting (or identifying) the transgenic plant of part (b) with enhanced mechanical stalk strength compared to a control plant not comprising the recombinant DNA construct.

A method of selecting for (or identifying) enhanced mechanical stalk strength in a plant, the method comprising: (a) obtaining a transgenic plant, wherein the transgenic plant comprises in its genome a recombinant DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO:1; or (ii) derived from SEQ ID NO:1 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant with enhanced mechanical stalk strength, when compared to a control plant not comprising the recombinant DNA construct.

A method of producing seed comprising any of the preceding methods, and further comprising obtaining seeds from said progeny plant, wherein said seeds comprise in their genome said recombinant DNA construct.

In any of the preceding methods or any other embodiments of methods of the present invention, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present invention, said regenerating step may comprise the following: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present invention, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant invention.

The introduction of recombinant DNA constructs of the present invention into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Cloning and Validation of Maize Bk4 Gene

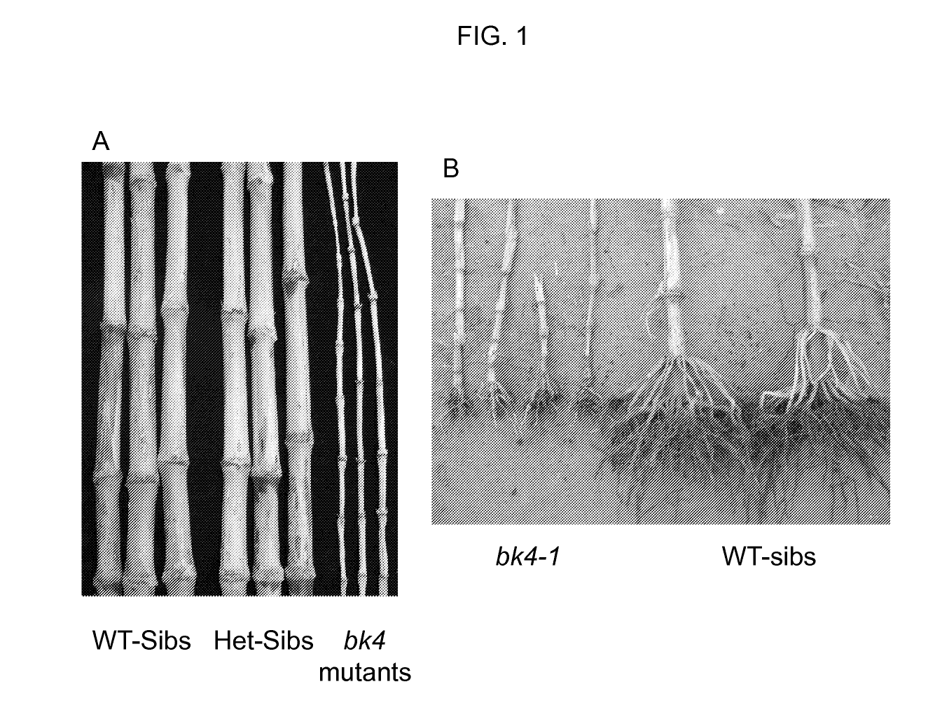

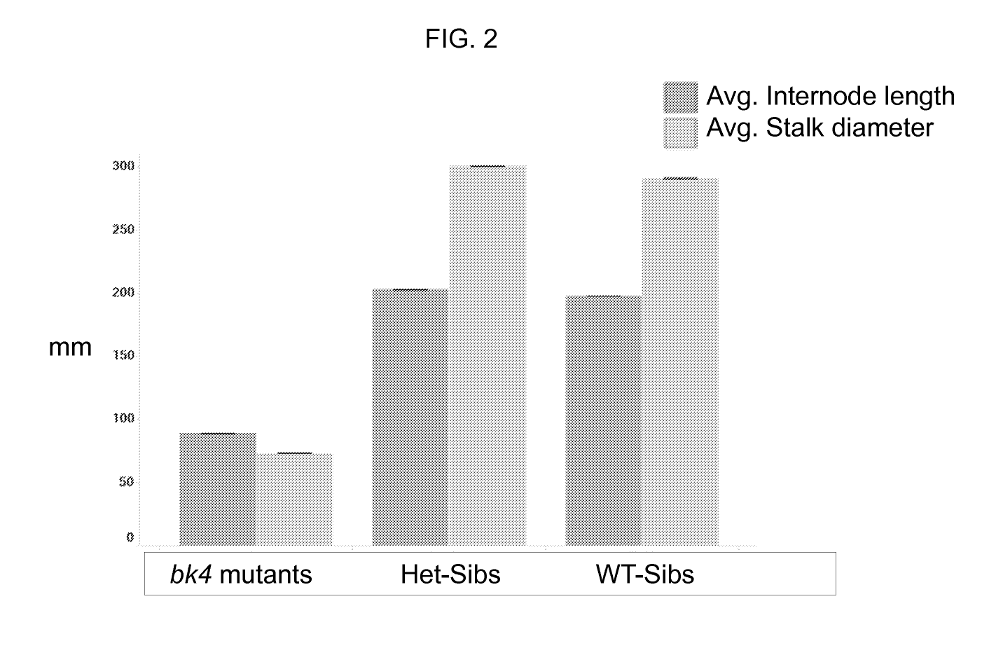

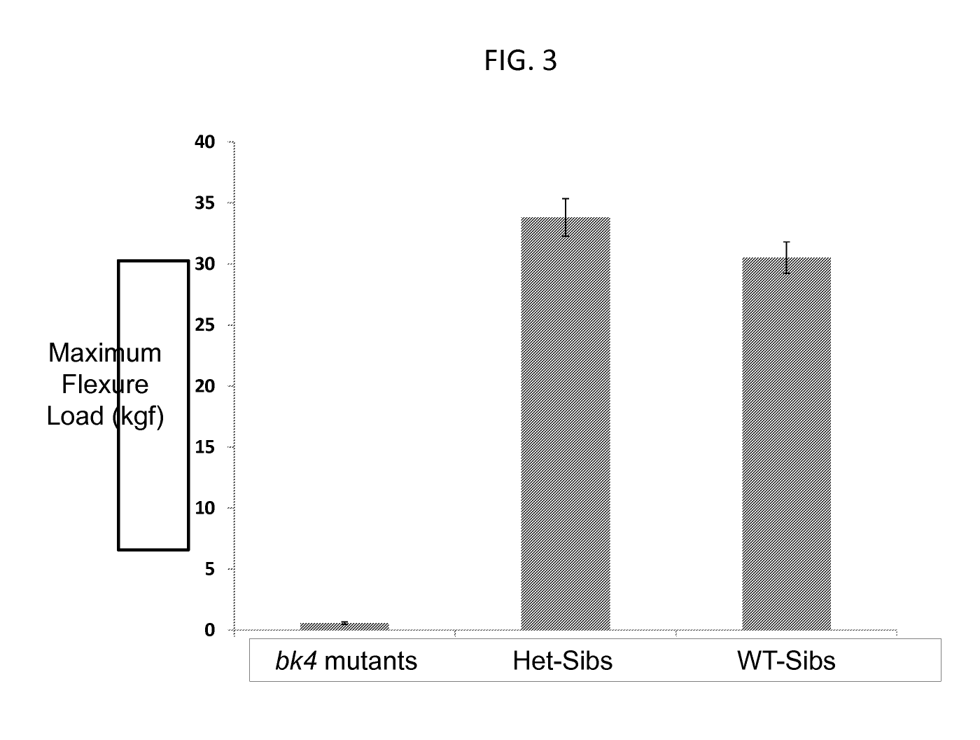

A brittle stalk mutant was identified from a self-population of a Mutator (Mu) x Inbred cross and was designated bk4. The bk4 homozygous mutants exhibited brittle plant parts including leaves, stalk, brace roots, midrib, and tassel (FIG. 1) and had shorter average internode length and decreased average stalk diameter (FIGS. 1 and 2). Moreover, the stalks of bk4 mutants exhibited little resistance to mechanical pressure (FIG. 3) as shown by assessing the mechanical or flexural strengths of wild-type and bk4 internodes using simple one-point bend tests. The internodes of wild-type plants continue to bend under increasing stress, but the internodes of bk4 mutant plants bend slightly and then snap upon continued applied stress.

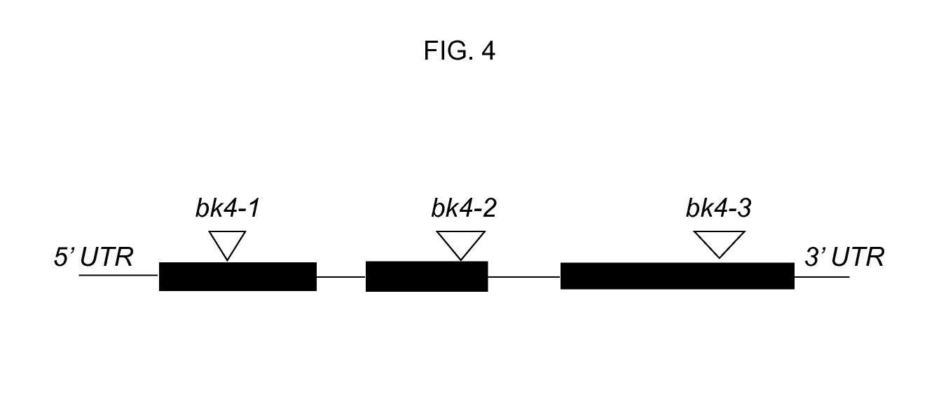

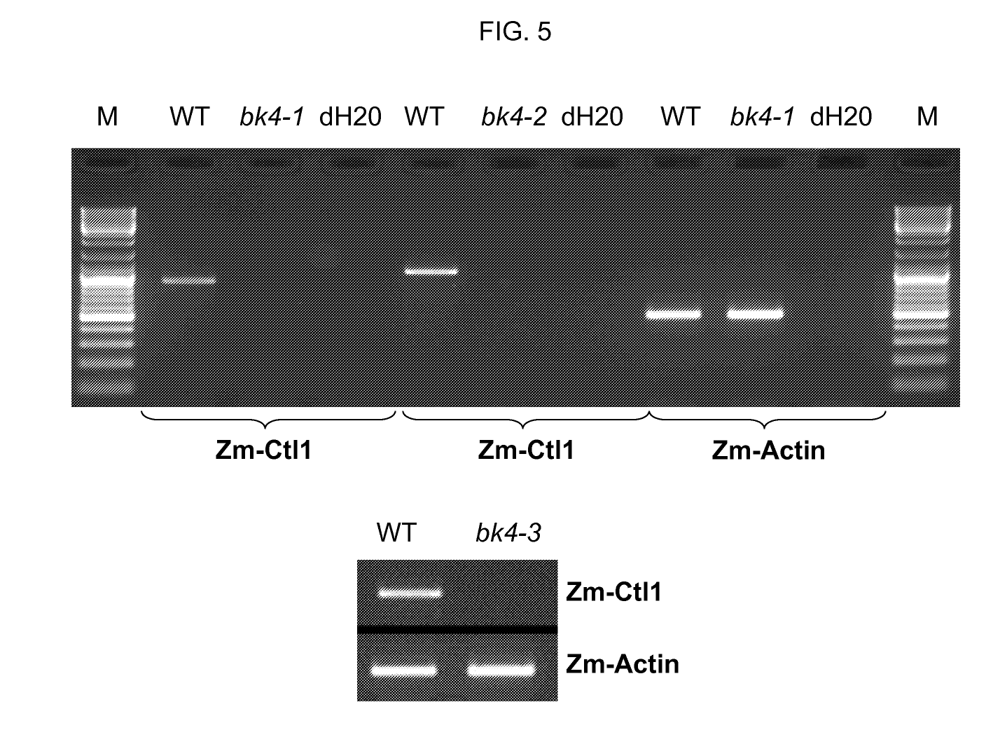

The mutant phenotype is due to a single recessive gene. The gene was cloned by co-segregation analysis with Mu, and it was determined that the Bk4 gene is on the long arm of chromosome 7 and encodes Chitinase-like protein 1 (ZmCTL1). The structure of the gene encoding the Chitinase-like protein 1 (ZmCTL1) is shown in FIG. 4. Two additional mutant alleles were also identified from the same population. Each allele has an insertion at a different site within the same gene (FIG. 4); however, all three alleles result in degradation of the mature transcript (FIG. 5). RT-PCR analysis using ten days old seedlings and gene specific primers showed missing transcripts in the homozygous mutants when compared to their WT-sibs (FIG. 5).

### Example 2

### Transcriptional Analysis of the Maize Bk4 Gene

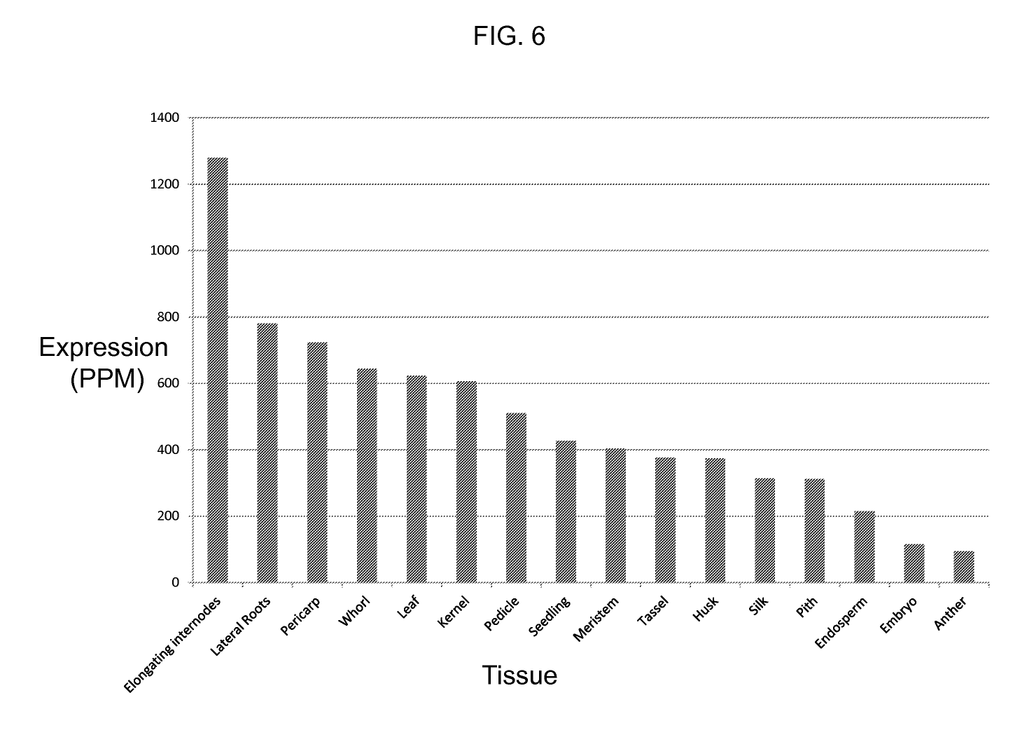

The expression pattern of the maize Ctl1 gene (FIG. 6) in different tissues of the inbred line B73 was assessed using massively parallel signature sequencing (MPSS) technology (Brenner et al. 2000. Nature Biotechnol. 18:630-634). ZmCtl1 is expressed at low level in seedlings (400 PPM) while its expression is approximately three-fold greater in elongating stalks at the V7-V8 stage of the plant (>1200 ppm). This preferential high expression is detected only in the mature zone of elongating internodes (9-10 cm above node) and specifically in vascular bundles isolated from rind tissue. Leaves and lateral roots at this stage have 40-50% less expression as compared to elongating internodes. The Ctl1 gene has the lowest expression in reproductive tissues (e.g. anther, embryo, endosperm, and silk) and in the pith of the stalk.

### Example 3

### Biochemical and Histochemical Analysis of bk4 Mutants as Compared to their WT-sibs

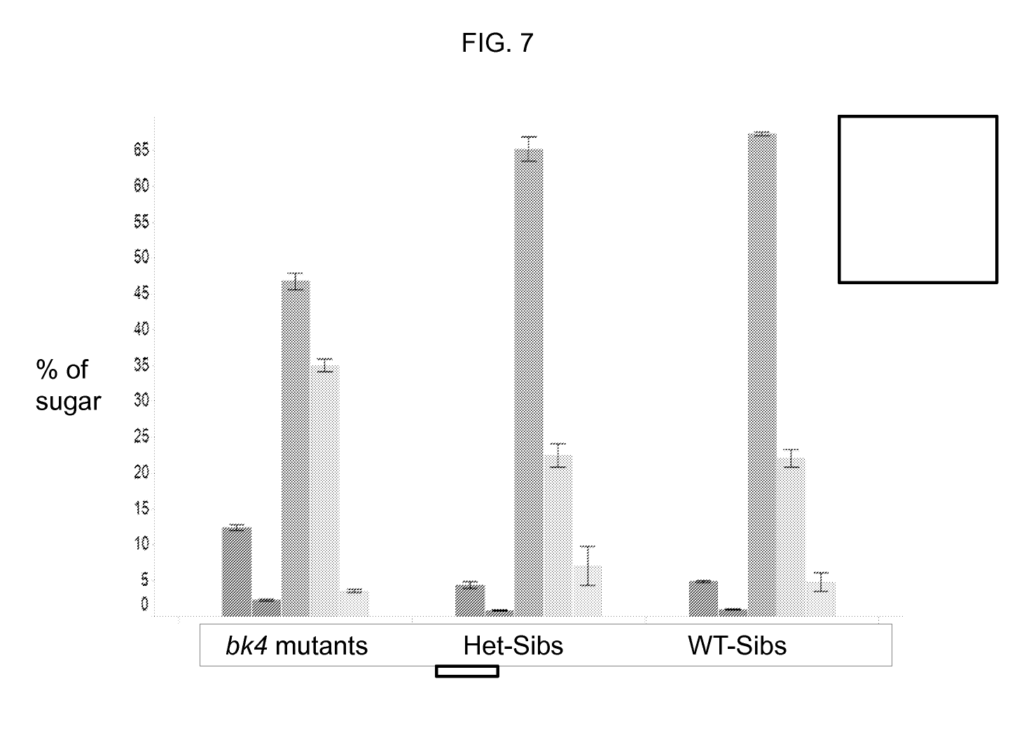

The stalks of bk4 homozygous mutants were assessed for differences in sugar compositions as compared to WT-sibs (FIG. 7). Arabinose, galactose, and xylose levels are higher in the mutants, while glucose is significantly reduced.

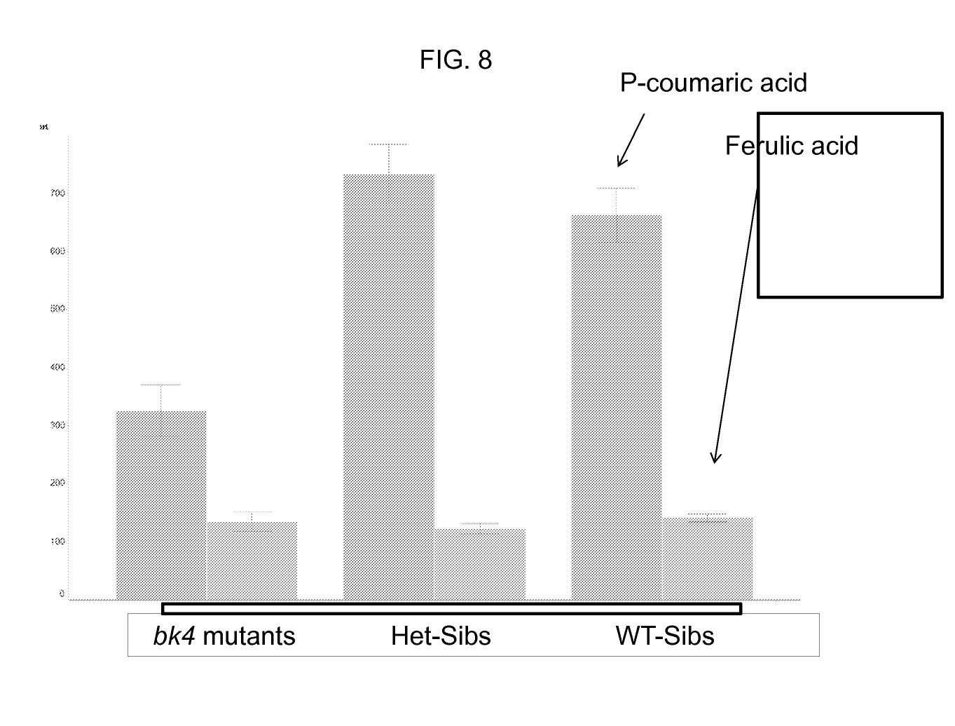

Levels of p-coumaric acid and ferulic acid were also examined in dried stalk tissue of bk4 mutants and WT-sibs. The bk4 mutants accumulate lower levels of p-coumaric acid (FIG. 8) in dried stalk tissue, while there is no significant difference between ferulic acid levels.

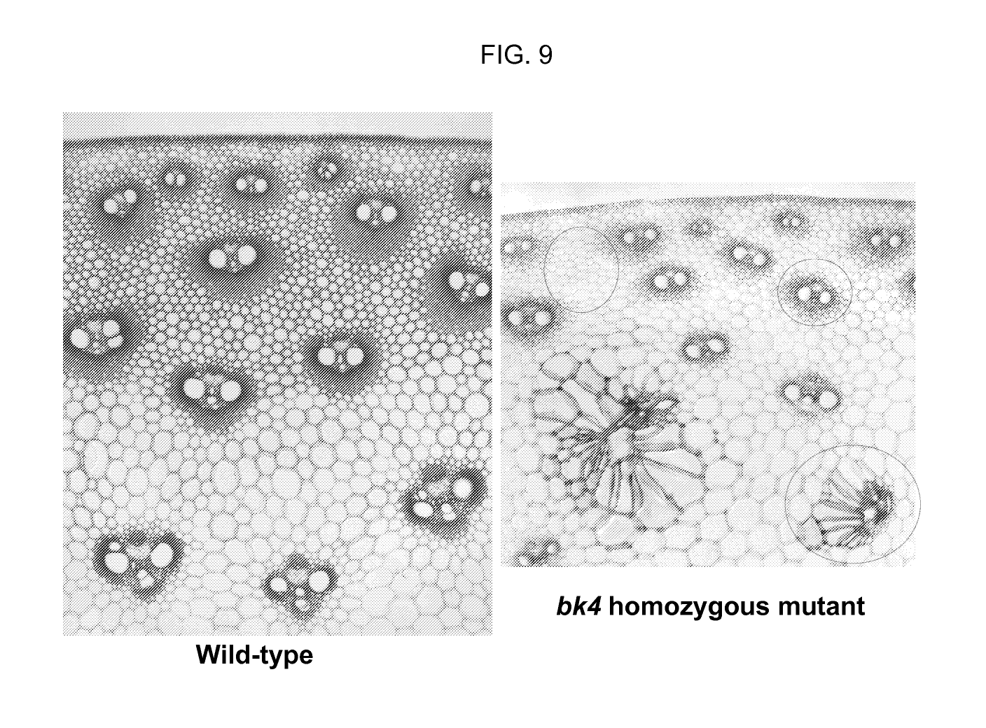

Lignins can be detected in tissue sections using specific stains such as the Maule reagent, acid fuchsin, and the Wiesner reagent (phloroglucinol). FIG. 9 shows phloroglucinol staining of stalk sections collected at the flowering stage of the plant. There is a significant reduction in lignin staining in the rind collenchyma cells and bundle fibers throughout the stem of bk4 mutants as compared to their WT-sibs and deformed bundles in the pith of bk4 mutants are common.

### Example 4

### Identification of Homologs of the Maize CTL1 Polypeptide

The maize CTL1 (BK4) polypeptide can be analyzed for similarity to all publicly available amino acid sequences contained in the “nr” database using the BLASTP algorithm provided by the National Center for Biotechnology Information (NCBI) as well as to the DUPONT™ proprietary internal databases.

A BLAST search using the sequence of the maize CTL1 polypeptide revealed similarity of the maize CTL1 polypeptide to chitinase-like proteins from various organisms. Shown in Table 5 (non-patent literature) and Table 6 (patent literature) are the BLASTP results for the amino acid sequence of the maize CTL1. Also shown in Tables 5 and 6 are the percent sequence identity values for each pair of amino acid sequences using the Clustal W method of alignment with default parameters:

TABLE 5
BLASTP Results for Maize CTL1 Polypeptide (Non-patent)

| NCBI Identifier | % Seq Identity |
|---|---|
| GI226500888 | 99.7 |
| (SEQ ID NO: 3) | |
| GI242045186 | 96.6 |
| (SEQ ID NO: 4) | |
| GI115479911 | 85.9 |
| (SEQ ID NO: 5) | |
| GI357159137 | 82.8 |
| (SEQ ID NO: 6) | |
| GI283046278 | 72.7 |
| (SEQ ID NO: 7) | |
| GI342151641 | 75.7 |
| (SEQ ID NO: 8) | |
| GI409191689 | 70.0 |
| (SEQ ID NO: 9) | |
| GI242082217 | 71.2 |
| (SEQ ID NO: 10) | |
| GI326529205 | 69.7 |
| (SEQ ID NO: 11) | |
| GI115477370 | 68.6 |
| (SEQ ID NO: 12) | |
| GI125562231 | 68.6 |
| (SEQ ID NO: 13) | |
| GI357502783 | 67.7 |
| (SEQ ID NO: 14) | |
| GI225431904 | 65.3 |
| (SEQ ID NO: 15) | |
| GI37051096 | 71.2 |
| (SEQ ID NO: 16) | |
| GI388492432 | 67.3 |
| (SEQ ID NO: 17) | |
| GI363807428 | 66.7 |
| (SEQ ID NO: 18) | |
| GI356526631 | 67.0 |
| (SEQ ID NO: 19) | |
| GI15221283 | 66.6 |
| (SEQ ID NO: 20) | |
| GI255549220 | 65.6 |
| (SEQ ID NO: 21) | |
| GI225897882 | 66.2 |
| (SEQ ID NO: 22) | |
| GI297848858 | 65.6 |
| (SEQ ID NO: 23) | |
| GI425886500 | 67.3 |
| (SEQ ID NO: 24) | |

TABLE 6
BLASTP Results for Maize CTL1 Polypeptide (Patent)

| | | | Percent |
|---|---|---|---|
| Sequence | Reference | BLASTP | Sequence |
| (SEQ ID NO) | (SEQ ID NO) | E-value* | Identity |
| ZmCTL1 | SEQ ID NO: 16 in | 1.57e−200 | 100 |
| (SEQ ID NO: 3) | WO2005011366; | | |
| | SEQ ID NO: 12 in | | |
| | US2003010184, | | |
| | WO0056908, and | | |
| | U.S. Pat. No. 6,563,020 | | |

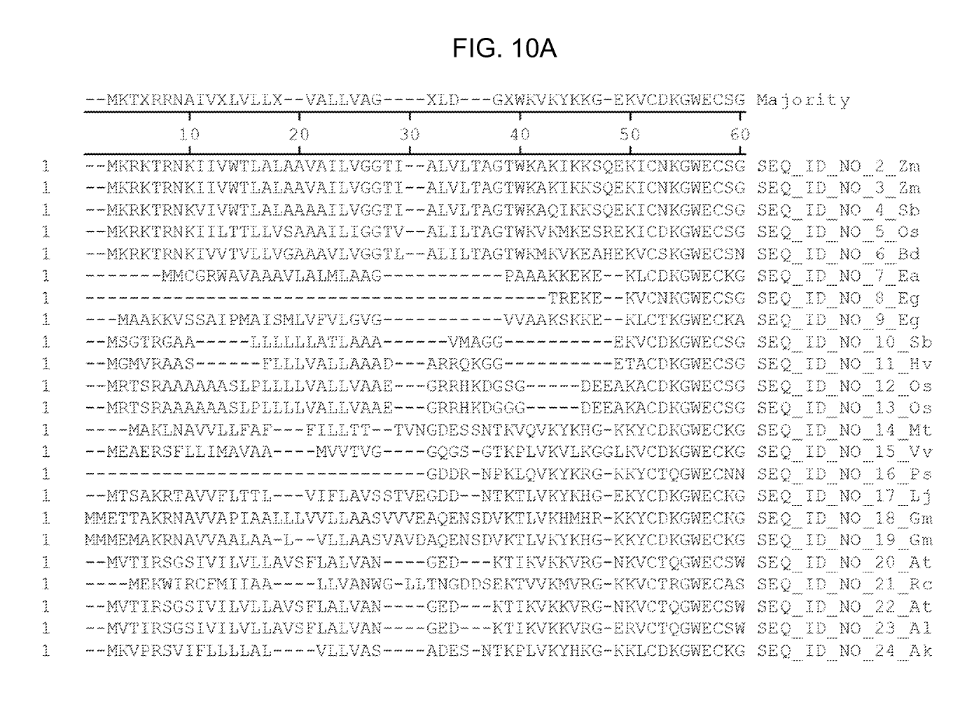

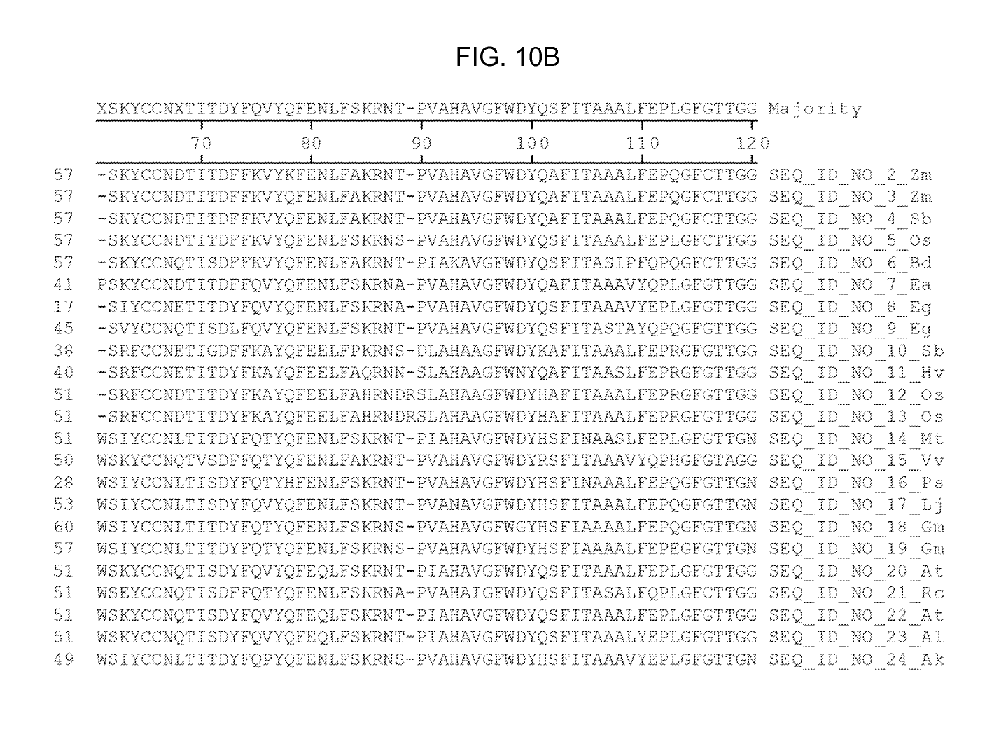

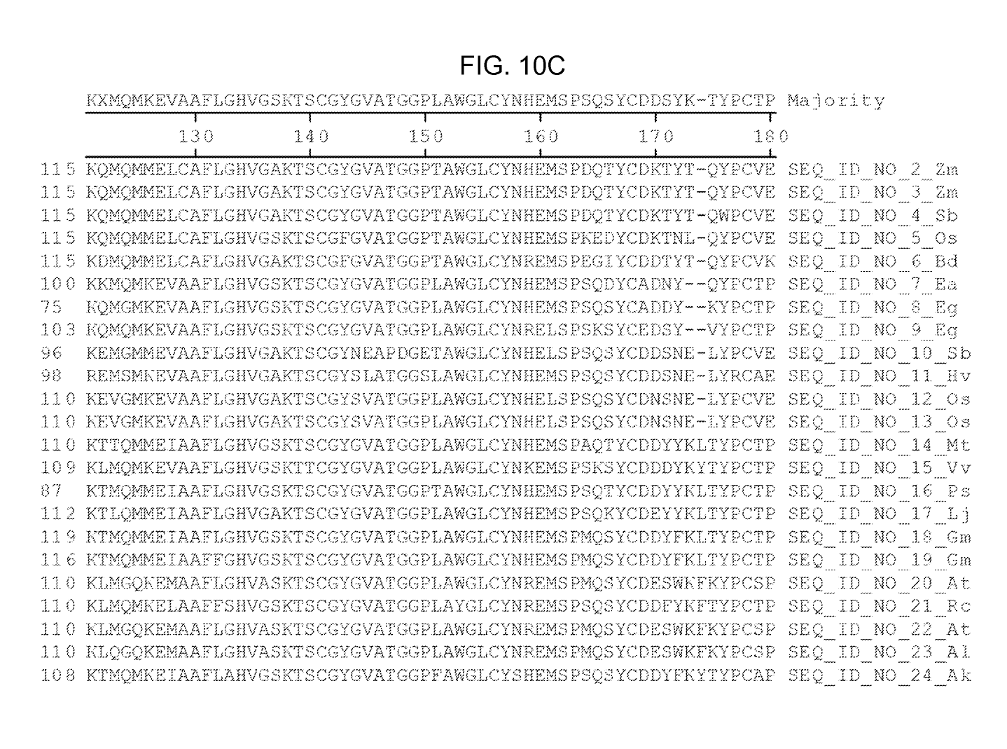

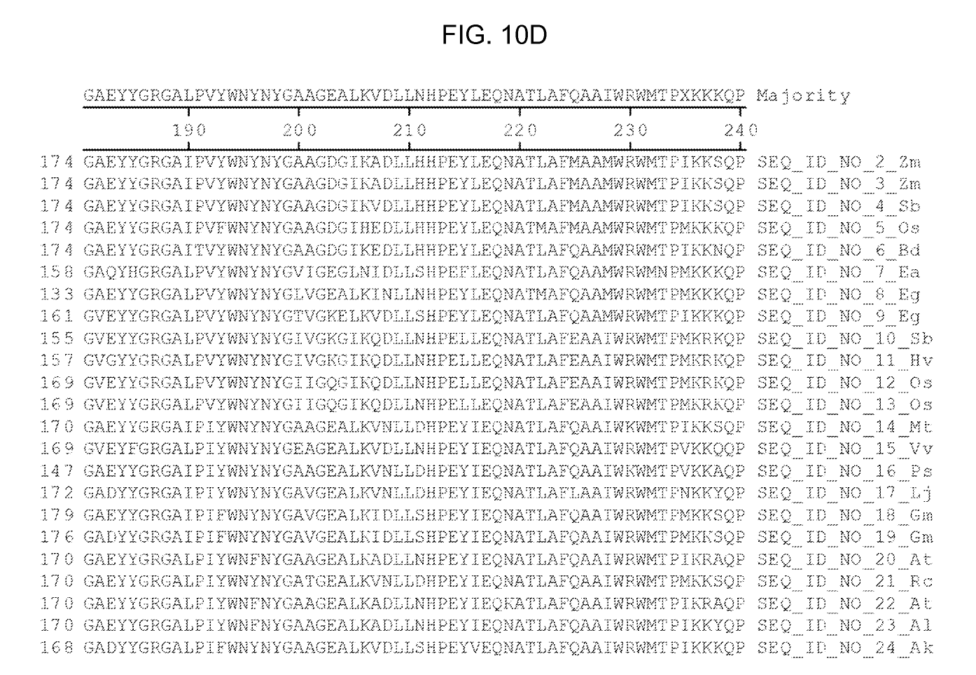

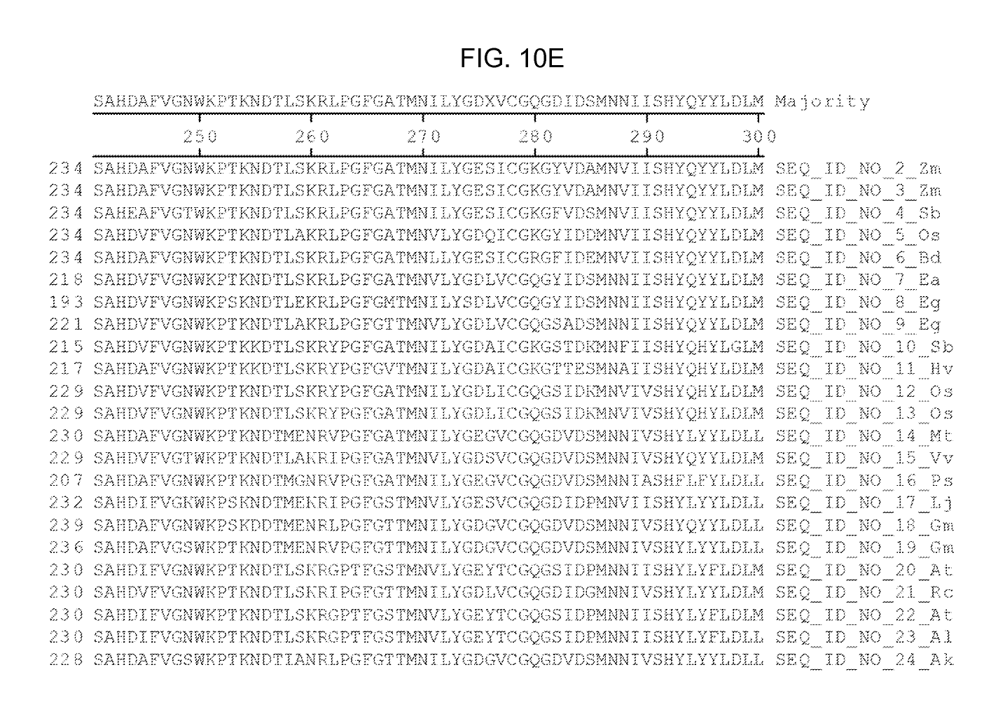

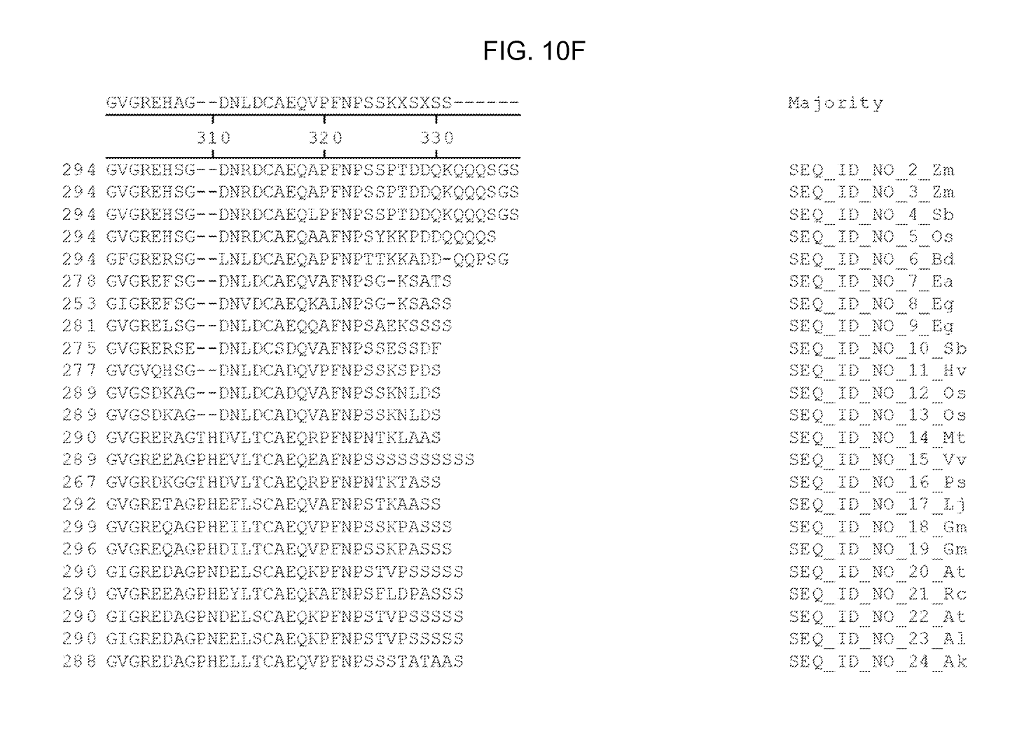

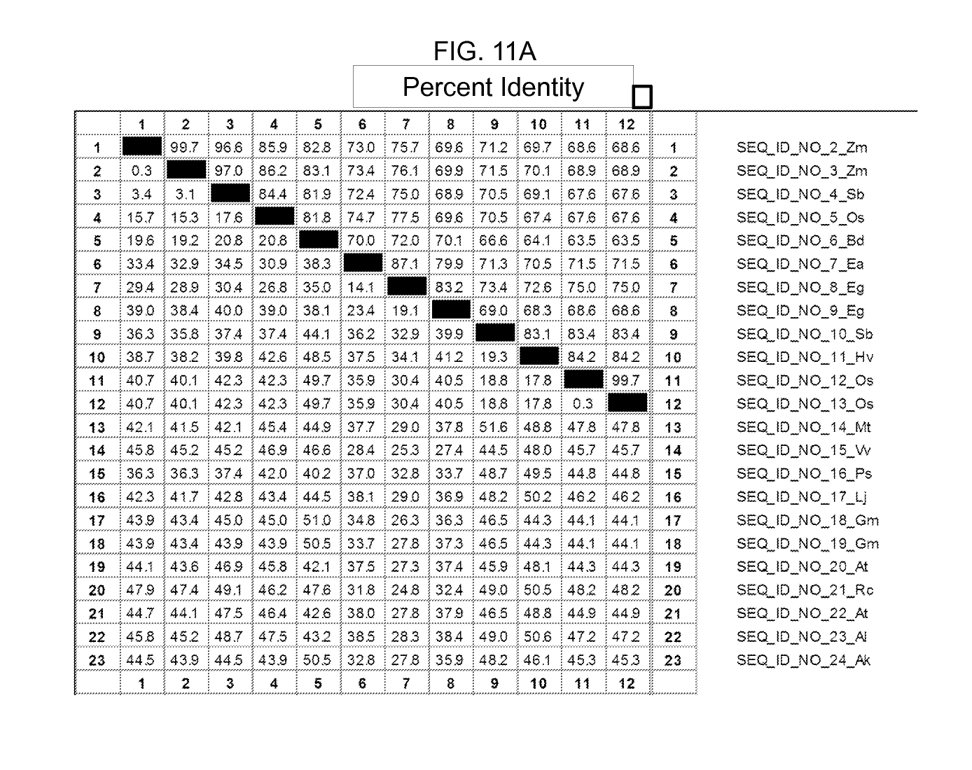

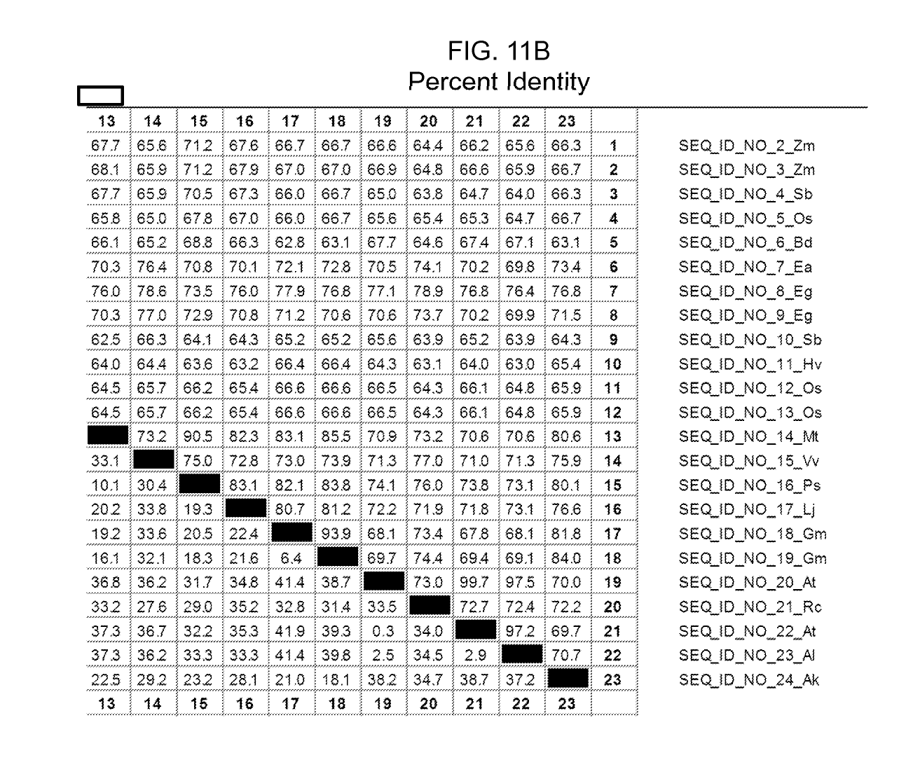

FIGS. 10A-10F present an alignment of the amino acid sequences of the polypeptides set forth in SEQ ID NOs:3-24. FIGS. 11A and 11B present the percent sequence identities and divergence values for each sequence pair presented in FIGS. 10A-10F.

Sequence alignments and percent identity calculations were performed using the Megalign® program of the LASERGENE® bioinformatics computing suite (DNASTAR® Inc., Madison, Wis.). Multiple alignment of the sequences was performed using the Clustal W method of alignment (Thompson et al. (1994) Nucleic Acids Research. 22:4673-80) with the default parameters (GAP PENALTY=10, GAP LENGTH PENALTY=0.20). Default parameters for pairwise alignments using the Clustal method were GAP PENALTY=10.00 and GAP LENGTH=0.10. The Protein Weight Matrix used was the Gonnet series.

### Example 5

### Overexpressinq Ctl1 in Plants

The maize Ctl1 gene or any of its homologs can be inserted into a vector, which can further be transformed into plants (including but not limited to maize) using methods known to one of ordinary skill in the art. Phenotypic analysis can then be performed using any known method of assessment to determine the plant's mechanical stalk strength.

### Example 6

### Overexpression of Ctl1 in Maize Plants

A 1.6 kb fragment containing ctl1 was amplified from maize genomic DNA. The fragment was cloned into an entry clone, consisting of an enhanced maize ubiquitin promoter (plus 5′ UTR and intron), the Ctl1 coding region, and the PINII terminator. The entire cassette, surrounded by Gateway attL1 and attL2 recombination sites, was mobilized into the appropriate plant expression destination vector via an LR recombination reaction. The resultant Ubi-ctl1 construct, PHP44151, was introduced via Agrobacterium-mediated transformation into maize callus. Plants were regenerated from the callus, and three events were shown to have the full length transcript.

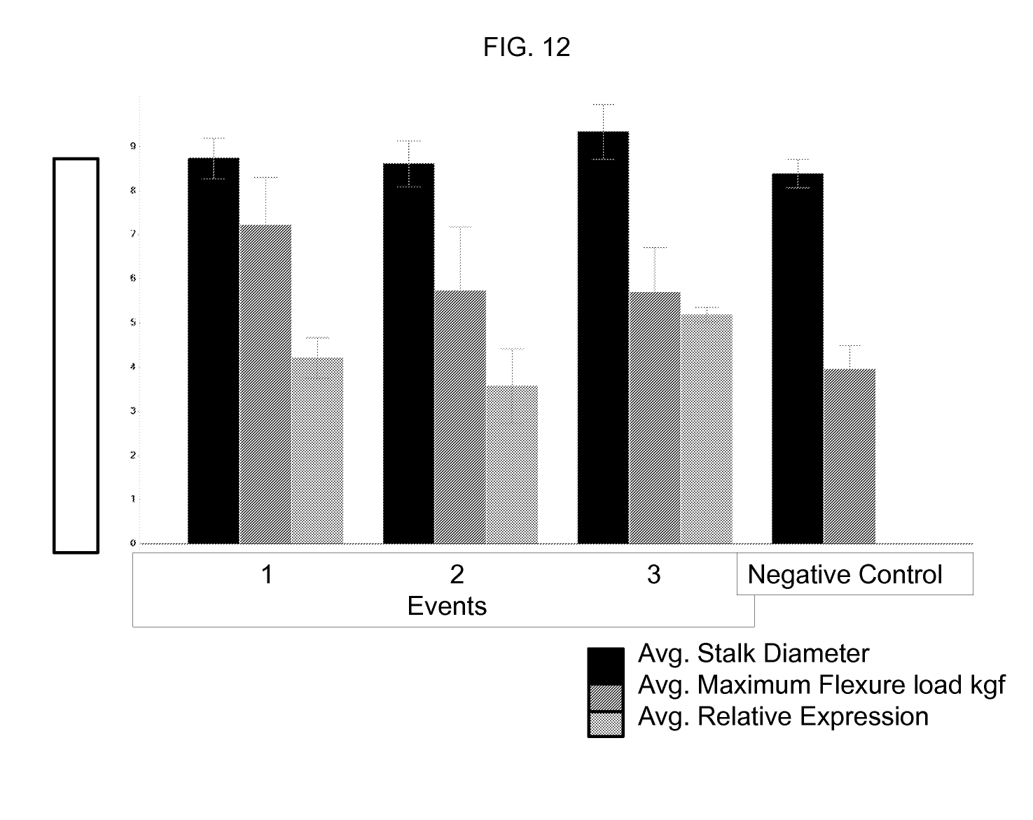

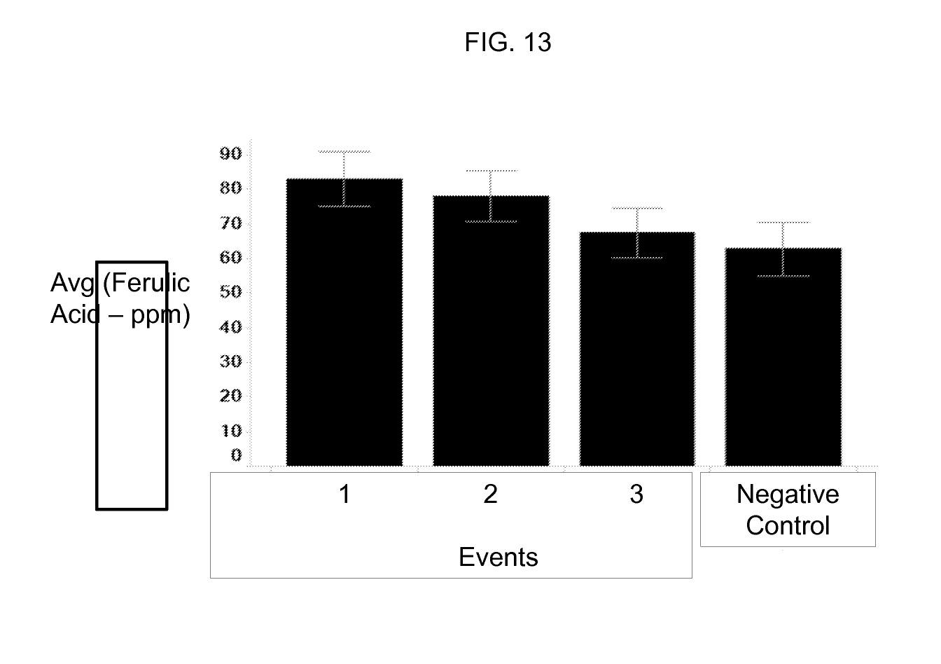

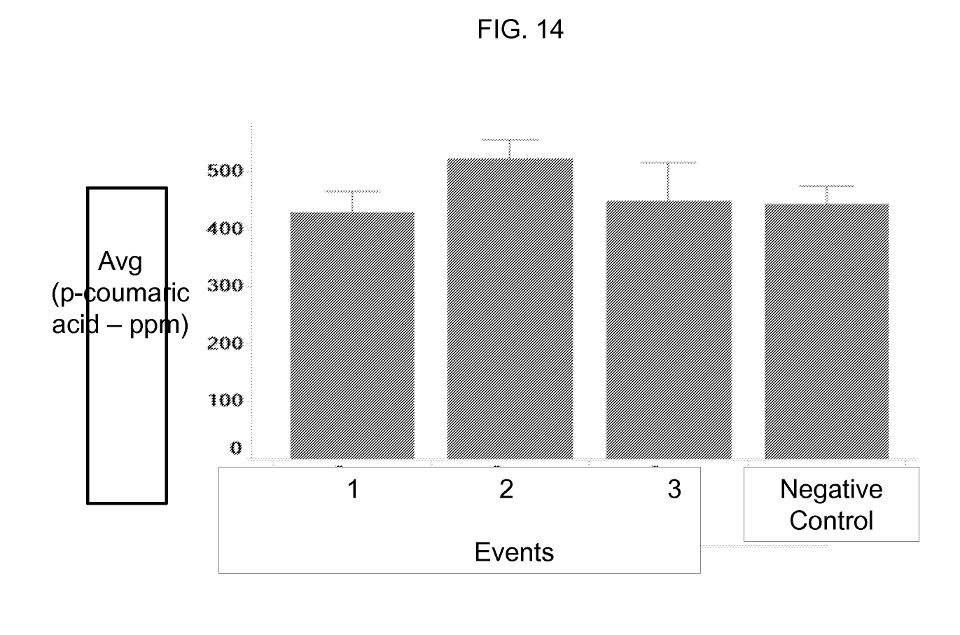

Overexpression of ZmCtl1 increased mechanical stalk strength (maximum flexure load kgf) and ferulic acid content significantly in event 1 and relatively in events 2 and 3 as compared to the negative control (FIG. 12 and FIG. 13), without affecting stalk diameter (FIG. 12) and p-coumaric acid content (FIG. 14), as assessed using T1 plants. These results are aligned with the levels of Ctl1 gene expression in events containing the transgene as compared to the negative control (FIG. 12).

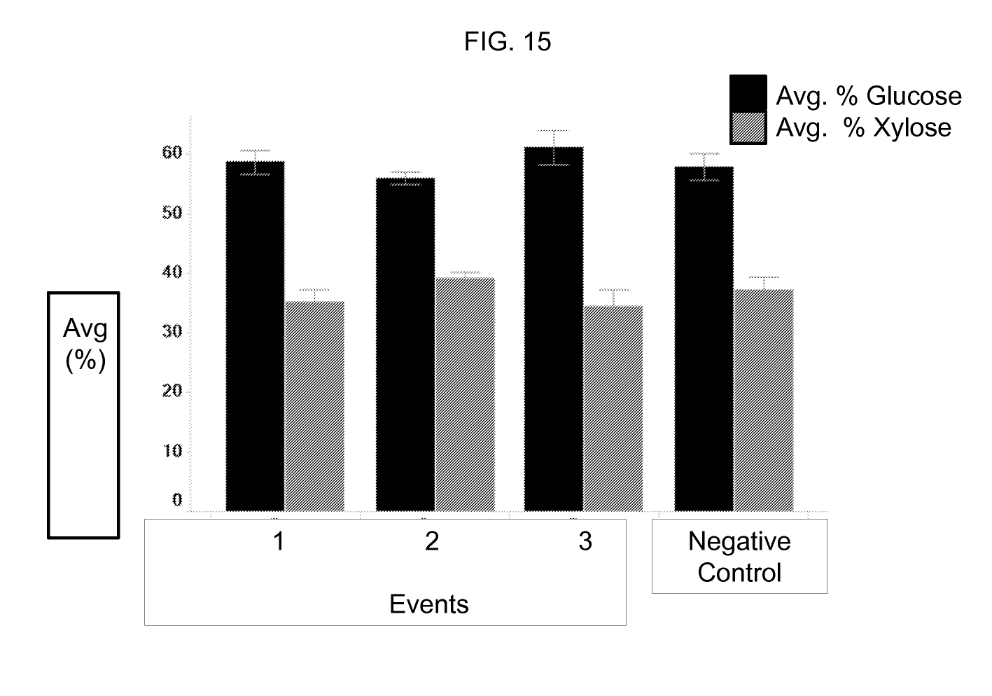

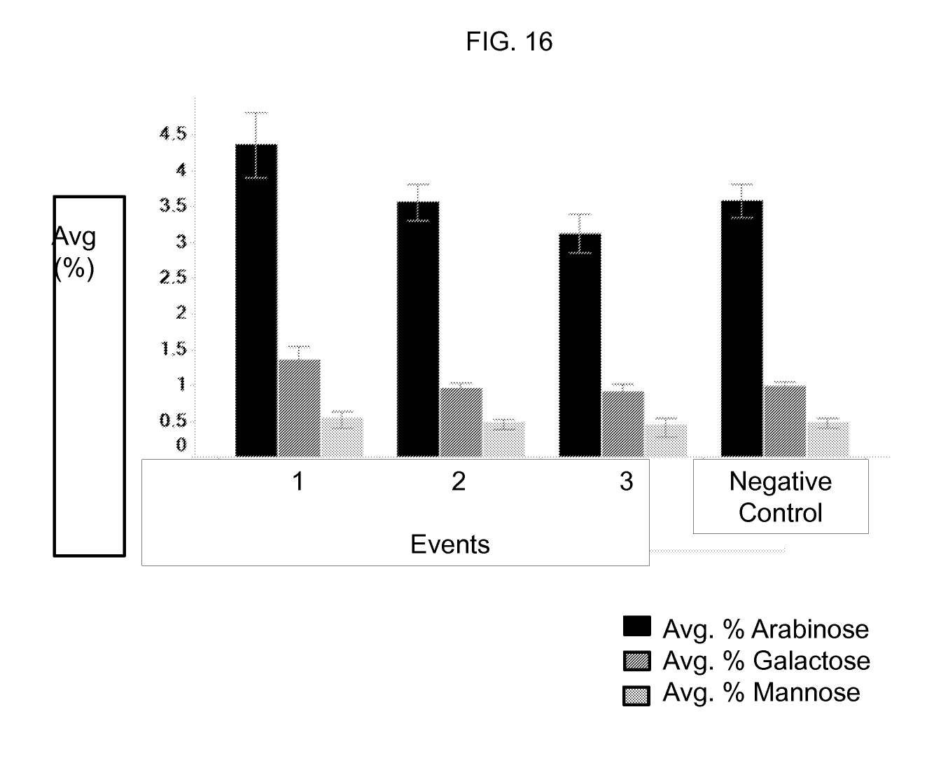

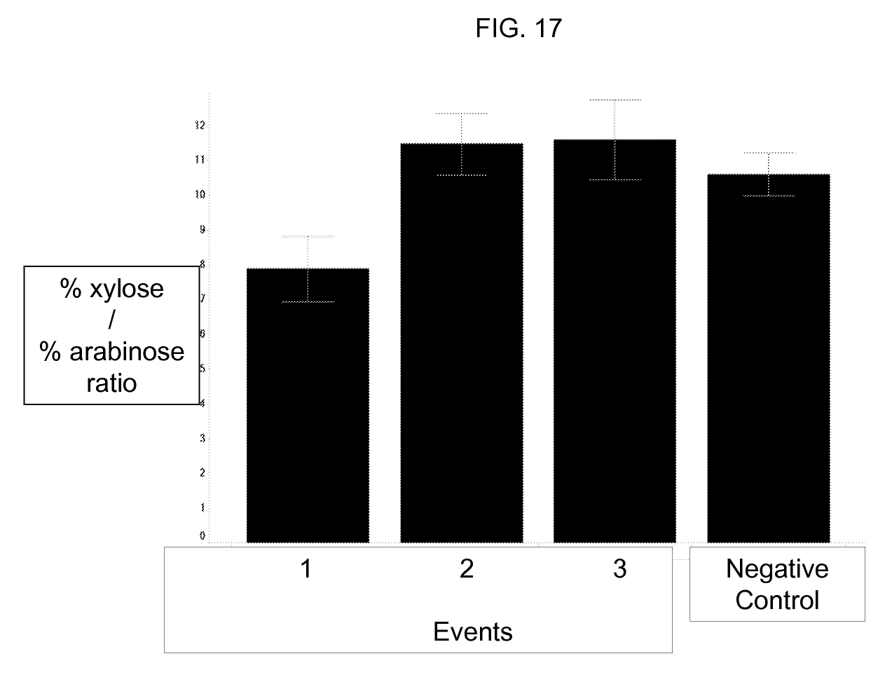

Additional analysis of the T1 plants showed minimal variations in the average percentage of glucose and a slight decrease in xylose content, particularly in event 1, as compared to the negative control (FIG. 15). The average percentage of arabinose and the average percentage of galactose were significantly higher in event 1 (FIG. 16), which led to a significant change in the ratio of xylose to arabinose in event 1 (FIG. 17).

The results indicate that the overexpression of ZmCtl1 is enhancing mechanical stalk strength by increasing only ferulic acid and arabinose content, which form cross-links in the lignin biosynthesis pathway. Furthermore, the overexpression of ZmCtl1 has no pleiotropic effect on other traits, such as sugars (glucose and mannose), p-coumaric acid, and stalk diameter in transgenic plants.

What is claimed is:

1. A plant comprising in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24, and wherein said plant exhibits enhanced mechanical stalk strength when compared to a control plant not comprising said recombinant DNA construct.

2. The plant of claim 1, wherein said plant is selected from the group consisting of: Arabidopsis, maize, soybean, sunflower, sorghum, canola, wheat, alfalfa, cotton, rice, barley, millet, sugar cane and switchgrass.

3. Seed of the plant of claim 1 or 2, wherein said seed comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24, and wherein a plant produced from said seed exhibits enhanced mechanical stalk strength when compared to a control plant not comprising said recombinant DNA construct.

4. A method of enhancing mechanical stalk strength in a plant, comprising:
(a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24;
(b) regenerating a transgenic plant from the regenerable plant cell of (a), wherein the transgenic plant comprises in its genome the recombinant DNA construct; and
(c) obtaining a progeny plant derived from the transgenic plant of (b), wherein said progeny plant comprises in its genome the recombinant DNA construct and exhibits enhanced mechanical stalk strength when compared to a control plant not comprising the recombinant DNA construct.

5. A method of selecting for enhanced mechanical stalk strength in a plant, comprising:
(a) obtaining a transgenic plant, wherein the transgenic plant comprises in its genome a recombinant DNA construct comprising a polynucleotide operably linked to at least one regulatory element, wherein said polynucleotide encodes a polypeptide having an amino acid sequence of at least 50% sequence identity, based on the Clustal V method of alignment, when compared to SEQ ID NO: 2, 3, 4, 5, 6, 7, 8, 9, 10, 11, 12, 13, 14, 15, 16, 17, 18, 19, 20, 21, 22, 23, or 24;
(b) growing the transgenic plant of part (a); and
(c) selecting the transgenic plant of part (b) with enhanced mechanical stalk strength compared to a control plant not comprising the recombinant DNA construct.

6. The method of claim 4 or 5, wherein said plant is selected from the group consisting of: Arabidopsis, maize, soybean, sunflower, sorghum, canola, wheat, alfalfa, cotton, rice, barley, millet, sugar cane and switchgrass.
